(19) United States
(12) Patent Application Publication
(10) Pub. No.: US 2025/0049843 A1
(43) Pub. Date: Feb. 13, 2025

(54) IRON OXIDE NANOPARTICLE-MEDIATED RADIATION DELIVERY FOR TARGETED CANCER TREATMENT

(71) Applicant: University of Washington, Seattle, WA (US)

(72) Inventors: Miqin ZHANG, Seattle, WA (US); Peter A. CHIARELLI, Seattle, WA (US); Richard REVIA, Seattle, WA (US); Zachary STEPHEN, Seattle, WA (US); Forrest M. KIEVIT, Seattle, WA (US); Kui WANG, Seattle, WA (US); Richard G. ELLENBOGEN, Seattle, WA (US)

(73) Assignee: University of Washington, Seattle, WA (US)

(21) Appl. No.: 18/723,132

(22) PCT Filed: Dec. 21, 2022
(86) PCT No.: PCT/US2022/053668

§ 371 Date: Jun. 21, 2024

Related U.S. Application Data
(60) Provisional application No. 63/293,597, filed on Dec. 23, 2021.

(51) Int. Cl. A61K 33/26; A61K 41/00; A61N 5/10; B82Y 5/00

(52) U.S. Cl. CPC A61K 33/26; A61K 41/0038; A61N 5/10; B82Y 5/00

(57) ABSTRACT

Method for nanoparticle-mediated deposition of radiation (NMDR) and targeted radiation therapies using a biodegradable and bioabsorbable iron oxide nanoparticle with a biocompatible coating that is effective to overcome various extra- and intra-cellular barriers and selectively accumulate in solid and metastatic tumors to improve the energy transfer of conventional radiotherapy

### CROSS-REFERENCE TO RELATED APPLICATION

This application claims the benefit of U.S. Patent Application No. 63/293,597, filed Dec. 23, 2021, expressly incorporated herein by reference in its entirety.

### STATEMENT OF GOVERNMENT LICENSE RIGHTS

This invention was made with government support under Grant No. R01 CA161953, awarded by the National Institutes of Health. The government has certain rights in the invention.

### BACKGROUND

Despite advances in the clinical options used to treat glioblastoma (GBM), including chemotherapy, surgical excision, radiotherapy, and tumor radiosensitization, the life expectancy for patients has changed little over the past decades. The introduction of radiotherapy for GBM was found to slow disease progression by about 2-fold, although average survival for this WHO grade 4 neoplasm remains 12-15 months. Challenges in treatment of GBM arise from regional heterogeneity of the blood-brain barrier (BBB) near tumor tissue, distant intracranial spread of cellular metastases past the enhancing tumor margin, and development of resistance to chemotherapy.

The treatment of GBM by radiation alone is hindered, in part, by (i) incomplete radiosensitivity of GBM cells, and (ii) detrimental effects of radiation on surrounding neuroglial tissue. External beam radiation (e.g., intensity modulated radiotherapy) is most commonly utilized for treatment, although generation of radiation damage (or linear energy transfer; LET) is haphazard, occurring both in tumor and in normal brain. Conventional radiation relies on rapid DNA turnover within tumor cells to confer selective damage.

Targeted nanoparticle (NP) research has resulted in the ability to reliably deliver nanoscaled clusters to tumor cells. NP-mediated deposition of radiation (NMDR)—also referred to as Auger therapy or nanoparticle-enhanced X-ray therapy—is the technique in which metallic NPs modify the effects of incident photon radiation. Materials with high atomic number (Z) have been well-explored for NMDR, with prior research utilizing heavy elements including silver, gadolinium, hafnium, and gold. Clinical translation of these heavy-metal based technologies can be hindered by limited biocompatibility. In contrast, iron oxide NPs possess distinct benefits over high-Z atom NPs, including approval by the U.S. Food and Drug Administration for blood pool imaging, known biocompatibility/excretion profiles in humans, and superparamagnetism that allows magnetic resonance imaging (MRI) visualization of NP within tumors. Despite these advantages, the application of iron oxide for NMDR has been minimally investigated to date, in large part due to its lower atomic number. The mechanism of iron oxide NPs on radiation enhancement and the implementation of intracranial tumor iron oxide NMDR remain unexplored.

A need exists for improved materials and methods for treating GBM and, more particularly, a need exists for a tumor-targeted NP formulation for intracranial NMDR. The present disclosure seeks to fulfill these needs and provides further related advantages.

### SUMMARY

The present disclosure provides methods for nanoparticle-mediated deposition of radiation (NMDR).

In one aspect, the disclosure provides a method for targeted radiation therapy in a subject that improves the energy transfer of conventional radiotherapy at a select site in a subject. In one embodiment, the method comprises:
(a) administering to a subject an iron oxide nanoparticle having an iron oxide core, a biocompatible coating surrounding the core, and a targeting agent associated with the coating, to provide a site in the subject having accumulated iron oxide nanoparticles; and
(b) applying γ- and/or x-ray irradiation to the subject at the site having accumulated iron oxide nanoparticles to produce photoelectrons at the site.

In another aspect, the disclosure provides a method for producing photoelectrons at a select site in a subject. In one embodiment, the method comprises:
(a) administering to a subject an iron oxide nanoparticle having an iron oxide core, a biocompatible coating surrounding the core, and a targeting agent associated with the coating, to provide a site in the subject having accumulated iron oxide nanoparticles; and
(b) applying γ- and/or x-ray irradiation to the subject at the site having accumulated iron oxide nanoparticles to produce photoelectrons at the site.

In a further aspect, the disclosure provides a method for treating a cancer in a subject. In one embodiment, the method comprises:
(a) administering to a subject an iron oxide nanoparticle having an iron oxide core, a biocompatible coating surrounding the core, and a targeting agent associated with the coating, to provide a site in the subject having accumulated iron oxide nanoparticles; and
(b) applying γ- and/or x-ray irradiation to the subject at the site having accumulated iron oxide nanoparticles to produce photoelectrons at the site, wherein the site is a cancerous tumor.

### DESCRIPTION OF THE DRAWINGS

The foregoing aspects and many of the attendant advantages of this disclosure will become more readily appreciated as the same become better understood by reference to the following detailed description, when taken in conjunction with the accompanying drawings.

FIGS. 1A-1D illustrate NP-mediated radiosensitization and NP size characterization. FIG. 1A is a schematic illustration emphasizing the increased focusing of energy deposition in tumor regions due to the absorption of ionizing radiation by metal NPs causing radiosensitization as compared to highly scattered energy deposition characterizing conventional radiotherapy. FIG. 1B is an illustrative representation of representative iron oxide NPs useful in the methods described herein (e.g., 5 polymer-coated iron oxide core NPs). FIG. 1C compares intensity-based hydrodynamic size distribution for representative synthesized NPs obtained by dynamic light scattering. FIG. 1D shows transmission electron microscopy images of each NP (scale bars=50 nm).

FIGS. 2A-2F illustrate NP-enhanced ROS generation. FIG. 2A is a schematic illustration of the four conditions considered in determining the NP candidates for radiosensitization. FIG. 2B compares measurement of therapeutic ROS generation as a function of NP concentration using the DCFH fluorescence assay; NPs were exposed to an 8 Gy dose of γ-irradiation at each concentration investigated. Results are expressed as percent change in fluorescence compared to post-radiation fluorescence of equivalent composition solution bearing 0 mM NPs. FIG. 2C compares the off-target ROS production for each NP formulation using the DCFH fluorescence assay with added H₂O₂ (no radiation applied). FIGS. 2D-2F compares the generation of therapeutic ROS as a function of radiation dose (top) or H₂O₂ dose (bottom) for NPCP (2D), IOSPM (2E), and MCP (2F) using deionized water as a control (grey). In each plot, a control curve demonstrates the fluorescence response of DCFH alone upon γ-radiation exposure. The difference in slope of the two lines is theorized to be proportional to the potency of the NMDR response. In FIGS. 2B-2F, all experiments were performed in triplicate (n=3 independent experiments) and data are shown as mean±s.d.

FIGS. 3A-3C shows in vitro evaluation of NPCP-mediated radiosensitization. FIG. 3A compares clonogenic survival as a function of radiation dose for SF767 cells incubated for 12 hours with NPCP at concentrations of 0, 25, or 100 μg Fe ml⁻¹. FIG. 3B illustrates fractional decrease in the number of cell colonies at a given radiation dose induced by incubation with NPCP at concentrations of 25 μg Fe ml⁻¹ or 100 μg Fe ml⁻¹ referred to the control case (cells not incubated with NPCP). FIG. 3C illustrates an Alamar blue assay using 50 μg ml⁻¹ of NPCP or NPCP-CTX as a function of γ-irradiation dose. The viability of cells exposed to NPs was normalized by viability of cells receiving no exposure to NPs, measured for each radiation dose. All experiments were performed in sextuplicate (n=6 independent experiments) and data are shown as the mean±s.d.

FIGS. 4A-4F show preferential NPCP-CTX uptake in tumor regions compared to NPCP without CTX. FIG. 4A compares pre-(left) and post-injection (right) MRI T₂*-weighted scans showing uptake of NPCP-CTX in tumor tissue. FIG. 4B compares pre-(left) and post-injection (right) T₂*-weighted images showing NPCPs in tumor tissue. FIG. 4C compares T₂*-weighted signal change from pre-injection baseline (expressed as a percentage) in tumor regions of interest (filled circles) and healthy brain regions of interest (unfilled circles) for a mouse injected with NPCP-CTX and a mouse injected with NPCP (FIG. 4D). The data are shown as mean±s.d. from the average of experiments performed in triplicate (n=3 independent experiments) where 100-voxel regions of interest were used to determine T₂*-weighted signal intensity normalized to the intensity of a water phantom. FIG. 4E illustrates T₂*-weighted signal change from pre-injection baseline (expressed as a percentage) in the external jugular vein. FIG. 4F illustrates tumor inoculation and treatment timeline. Radiotherapy was performed one hour after NPCP-CTX administration.

FIGS. 5A-5I illustrate increased survival by administration of NPCP-CTX and γ-irradiation. FIG. 5A illustrates representative T₂-weighted MRI scans emphasizing the size progression of tumors for each treatment group. FIG. 5B is a graph of mean tumor volume versus time for each treatment group (n=3 mice per group). Data are shown as mean±s.d. FIG. 5C is a Kaplan-Meier survival curve. FIG. 5D compares median survival for each treatment group. Statistical analysis was performed using the log rank test. In FIGS. 5C and 5D, n=17, 14, 9 and 12 for the untreated control, NPCP-CTX control, 10 Gy γ-irradiation control and combined NPCP+10 Gy γ-irradiation treatment groups, respectively. FIG. 5E compares representative histological (H&E) and IHC (Ki67 and γH2AX) tumor sections of orthotopic GBM implants in mice for treatment and control conditions. Scale bar represents 100 μm for all panels. FIGS. 5F-5I is a graphical violin plot representation comparing Ki67 and gH2AX fractional positivity (expressed as labeling index) and weighted histopathological scoring (H-score) quantified from ten random fields of view selected from each group including untreated, NPCP-CTX, radiation (10 Gy) and NPCP-CTX+10 Gy treatment.

FIGS. 6A-6D compare magnetic resonance spectra of normal and tumor tissue. FIGS. 6A and 6B illustrate magnetic resonance spectra acquired in healthy and tumorous regions of interest. Inset images presented show choice of region of interest in tumor and contralateral brain. FIGS. 6C and 6D show the results from the integration of the lactate peaks and the ratio of the integration of lactate-to-creatine peaks for the spectra shown in FIG. 6A, respectively.

### DETAILED DESCRIPTION

Radiotherapy is a mainstay adjunctive therapy for glioblastoma (GBM). Despite the outcome improvement achieved with radiation, GBM prognosis remains dismal. The present disclosure provides a tumor-targeted iron oxide nanoparticle (NP) that intensifies the energy transfer of conventional photon radiotherapy on a selective cellular basis. As described herein, nanoparticles were formulated with systematic architectural variation to optimize reactive oxygen species (ROS) production. A representative biocompatible tumor-targeted nanoparticle was tested in vitro using two models of GBM, and then in vivo, using an orthotopic human primary GBM xenograft mouse model. Animals that received intravenous NP before irradiation demonstrated a 3-fold reduction in tumor growth and a 2-fold increase in survival. Cellular damage was investigated using in vivo magnetic resonance spectroscopy, which demonstrated increased therapeutic cytotoxicity specific to the tumor mass. The present disclosure provides a viable therapeutic strategy to improve radiation therapy for GBM.

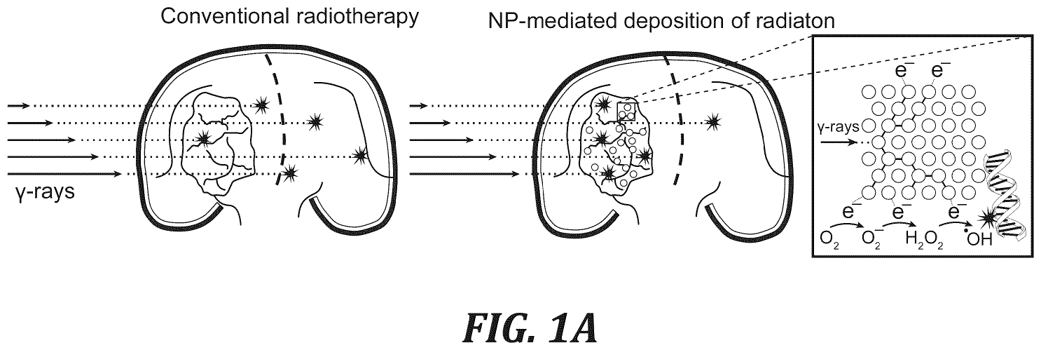

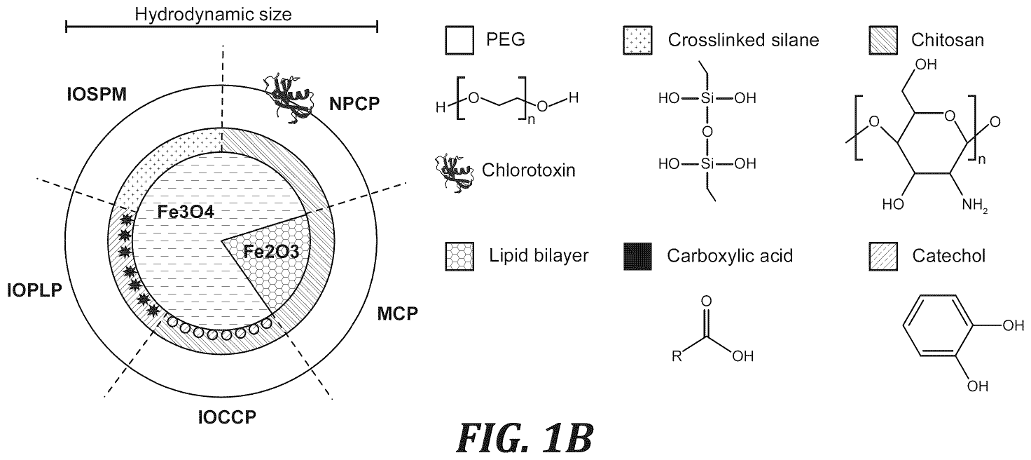

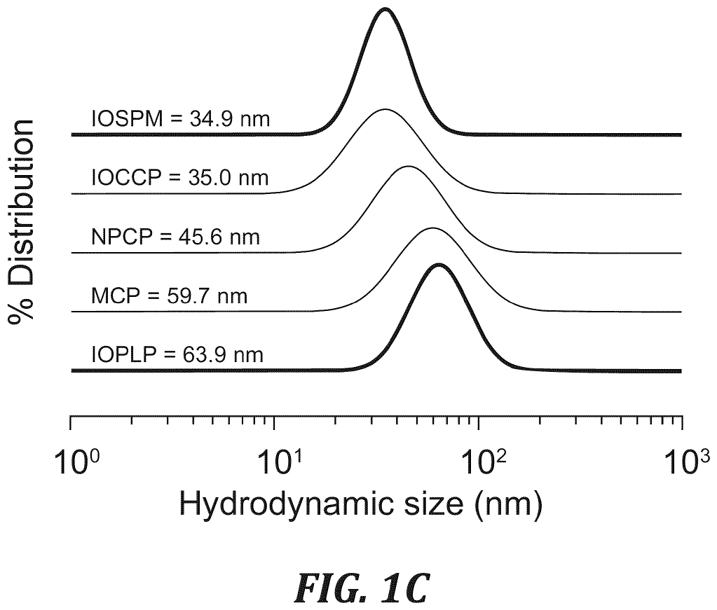

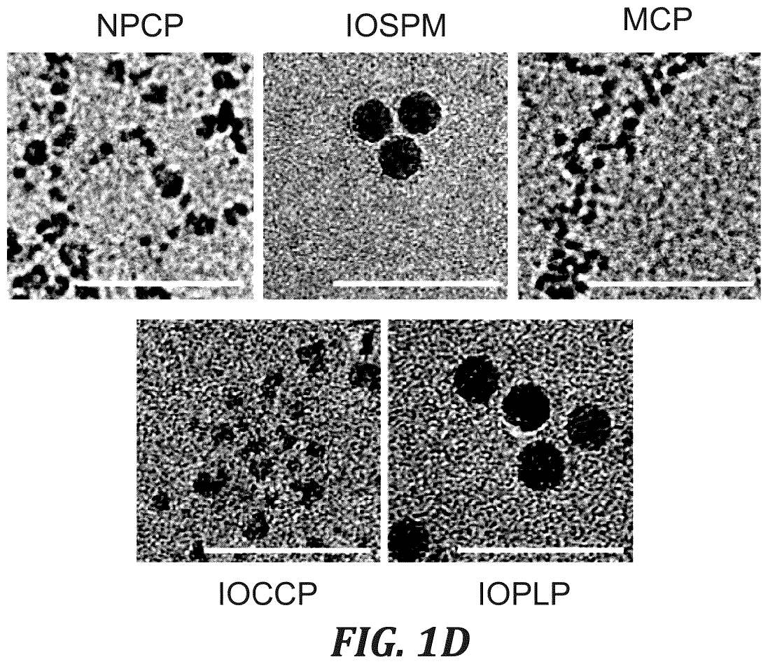

As described herein, the present disclosure exploits an innovation of the classic radiotherapy model. In addition to cell turnover as a source of tumor selectivity, the tumor-homing NP itself collects photon radiation, and changes its deposition pattern. The NP does not carry a therapeutic drug, but instead acts as a nanoscaled lens to gather incident photons and emit Auger photoelectrons. The use of NPs produces free-radical induced damage to tumor cells comparable to high-LET particle beam radiation but provides tumor specificity to minimize off-target damage (see FIG. 1A).

The methods of the present disclosure are photo radiotherapeutic methods that improve conventional radiotherapy. The improvement is the result of the use of the iron oxide nanoparticles described herein. Conventional radiotherapy does not use these nanoparticles and therefore does not offer the advantages provided by the nanoparticles described herein, which enhance energy transfer from incoming radiation (γ- and/or x-ray irradiation) relative to conventional radiotherapies. Conventional radiotherapy only uses γ- and/or x-ray irradiation and does not involve any nanoparticles. Although phototherapy uses nanoparticles, heavier metal nanoparticles, such as gold-, silver-, gadolinium-, hafnium-core nanoparticles, which have been reported for radiotherapy enhancement, heavy-metal nanoparticles are toxic and non-biodegradable hindering their uses in the clinic. The use of the biocompatible and biodegradable iron oxide nanoparticles described herein can be readily used in the clinical setting, given the unique degradability and resorbability of iron oxide nanoparticles, and FDA approval of certain iron oxide-core nanoparticles.

In the practice of the method of the disclosure, incoming irradiation to the nanoparticles accumulated at the site results in the formation of photoelectrons at the site and these photoelectrons are responsible for cell killing. Auger photoelectrons are among the photoelectrons produced by the method.

In one aspect, the disclosure provides a method for targeted radiation therapy in a subject that improves the energy transfer of conventional radiotherapy at a select site in a subject. In one embodiment, the method comprises:
(a) administering to a subject an iron oxide nanoparticle having an iron oxide core, a biocompatible coating surrounding the core, and a targeting agent associated with the coating, to provide a site in the subject having accumulated iron oxide nanoparticles; and
(b) applying γ- and/or x-ray irradiation to the subject at the site having accumulated iron oxide nanoparticles to produce photoelectrons at the site.

In another aspect, the disclosure provides a method for producing photoelectrons at a select site in a subject. In one embodiment, the method comprises:
(a) administering to a subject an iron oxide nanoparticle having an iron oxide core, a biocompatible coating surrounding the core, and a targeting agent associated with the coating, to provide a site in the subject having accumulated iron oxide nanoparticles; and
(b) applying γ- and/or x-ray irradiation to the subject at the site having accumulated iron oxide nanoparticles to produce photoelectrons at the site.

In a further aspect, the disclosure provides a method for treating a cancer in a subject. In one embodiment, the method comprises:
(a) administering to a subject an iron oxide nanoparticle having an iron oxide core, a biocompatible coating surrounding the core, and a targeting agent associated with the coating, to provide a site in the subject having accumulated iron oxide nanoparticles; and
(b) applying γ- and/or x-ray irradiation to the subject at the site having accumulated iron oxide nanoparticles to produce photoelectrons at the site, wherein the site is a cancerous tumor.

The present disclosure provides methods for nanoparticle-mediated deposition of radiation (NMDR). The methods described herein are targeted radiation therapies that effectively improve the energy transfer of conventional radiotherapy. The improvement in these methods is the administered iron oxide nanoparticle.

As described herein, the iron oxide nanoparticle has (i) a core that functions as a radiosensitizer that upon irradiation produces reactive oxygen species (ROS) that have the effect of cell killing in the environment of the localized nanoparticle, (ii) a biocompatible coating surrounding the core that is biodegradable and provides for increased cellular uptake and tumor accumulation, and (iii) a targeting agent that assists in selectively delivering the nanoparticle to its intended site.

The components of the iron oxide nanoparticle render the nanoparticle nontoxic and safe for human administration. It is well known that the FDA has approved iron nanoparticles for human administration for therapeutic applications (Ferumoxytol (FERAHEME) is a polymer-coated iron oxide nanoparticle that has been approved for treating iron deficiency). The nanoparticle described herein is biocompatible due in part to its biodegradability to non-toxic and safe degradation products. It will be appreciated that the nanoparticle useful in the present methods does not include a conventional therapeutic agent, such as a chemotherapeutic agent traditionally used for cancer treatment. Accordingly, because the nanoparticle does not include such a therapeutic agent, the therapeutic activity associated with the nanoparticle is due to the iron component of the nanoparticle acting as a radiosensitizer. Because the nanoparticle does not include a therapeutic agent, the nanoparticle does not suffer from the same safety and toxicity concerns of such agents or nanoparticles that include such agents. As noted above, the nanoparticle is non-toxic and safe for human administration.

The nanoparticle provides high cellular uptake and accumulation in a tumor. First, by virtue of the targeting agent (e.g., chlorotoxin), the nanoparticle selects tumor cells for targeting and ultimately nanoparticle delivery to that cell. Second, by virtue of its coating (e.g., chitosan), the nanoparticle is relatively positively charged which electrostatically assists delivery to cancer cells, which tend toward having relatively low pH. Also, the nanoparticles useful in the methods are relatively small (e.g., nanoparticles diameters from about 5 to about 200 nm) and therefore have advantages not only for cellular uptake and accumulation but also for crossing the blood-brain barrier to reach brain cancers and for releasing more iron (i.e., radiosensitizer effectiveness) relative to larger-sized iron oxide nanoparticles.

The methods described herein are targeted radiation therapies that effectively improving the energy transfer of conventional radiotherapy and are useful for producing photoelectrons at a select site in a subject, which in turn provides selective methods for treating cancer.

In certain embodiments of the above methods, the photoelectrons are Auger photoelectrons.

In certain of the above methods, the targeting agent selectively delivers the iron oxide nanoparticle to the site. In certain embodiments, the targeting agent selectively delivers the iron oxide nanoparticle to the cancerous tumor. In certain embodiments, the targeting agent is chlorotoxin. Suitable targeting agents include any agent having an affinity to the site of treatment (e.g., cancer cell). Representative targeting agents include small organic molecules, peptides, aptamers, and proteins and protein fragments.

In the methods for treating cancer described herein, the cancerous tumor is a solid tumor. In certain embodiments, the cancerous tumor is a brain tumor of any pathology. In certain of these embodiments, the cancerous tumor is a primary brain tumor (adult or childhood) or a brain metastasis tumor (adult or childhood). Representative primary brain tumors treatable by the methods include glioblastoma multiform, meningioma, and ependymoma. In other of these embodiments, the cancerous tumor is a neuroectodermal tumor. Representative neuroectodermal treatable by the methods include medulloblastoma, neuroblastoma, ganglioneuroma, metastatic melanoma, primary melanoma, pheochromocytoma, Ewing's sarcoma, small cell lung carcinoma, and schwannoma. In further of these embodiments, the cancerous tumor is a tumor of the breast, kidney, liver, lung, lymphoma, ovary, pancreas, prostate, cervix, colon, throat or bone. Other cancers treatable by the methods include oral, skin, and blood cancers (e.g., leukemia).

In the methods described herein, the iron oxide nanoparticle is administered to the subject intravenously (e.g., injection or infusion using a pharmaceutically acceptable carrier).

In certain embodiments of the methods described herein, the iron oxide core comprises magnetite (e.g., biodegradable magnetite). Other useful core materials include ferrous oxide, ferric oxide, silicon oxide, polycrystalline silicon oxide, silicon nitride, aluminum oxide, germanium oxide, titanium, titanium dioxide, gold, and mixtures thereof

In certain embodiments, the coating is effective to disperse the iron oxide nanoparticles (prevent nanoparticle aggregation) and have thicknesses in the range of 1-100 nm. In certain embodiments, the coating comprises a silanized poly(ethylene glycol) (PEG) monolayer (IOSPM) or a chitosan-PEG (CP) copolymer (NPCP) layer. In other embodiments, the coating comprises carbon, graphene quantum dots, silicon, silicon oxide, glutamine peptides, polyethyleneimine (PEI), polylysine, polyarginine, DNAs, and siRNAs.

In certain embodiments, the nanoparticle has a diameter from about 5 to about 200 nm. In certain of these embodiments, the nanoparticles have a polydispersity index (DPI) from about 0.05 to about 0.2.

In certain embodiments, the iron oxide nanoparticle is as described in FIG. 1B.

In another aspect, the disclosure provides for the use of an iron oxide nanoparticle having an iron oxide core, a biocompatible coating surrounding the core, and a targeting agent associated with the coating, for nanoparticle-mediated deposition of radiation (NMDR) in a subject.

In a further aspect, the disclosure provides for the use of an iron oxide nanoparticle having an iron oxide core, a biocompatible coating surrounding the core, and a targeting agent associated with the coating, for treating a cancer in a subject.

The following is a description of the preparation and use of representative iron oxide nanoparticles in nanoparticle-mediated deposition of radiation for the treatment of cancer.

The NP-mediated generation of Auger photoelectrons, and subsequent damage to surrounding tumor by NMDR, depends on the chemical composition and size of the metal NP core, as well as on the architecture of the surrounding polymer shell. The ability to allow water and biomolecules in close proximity to the active core to facilitate ROS production by interaction with Auger electrons will vary based on shell conformation, hydrophobicity, and surface charge. Multiple NPs were synthesized with different iron oxide core size, redox state and polymer coating, as well as different hydrodynamic size (FIGS. 1B and 1C). Their potential to produce ROS upon exposure to T-irradiation was evaluated. The NPs chosen for this evaluation were prepared an iron oxide core. Variable coatings included (i) silanized poly(ethylene glycol) (PEG) monolayer (IOSPM), (ii) chitosan-PEG (CP) copolymer (NPCP), (iii) CP copolymer modified with catechol (IOCCP) and (iv) amphiphilic phospholipid-grafted PEG (IOPLP). A fifth NP was constructed with equivalent surface composition to NPCP (magnetite), but with a maghemite core particle (MCP). FIG. 1C illustrates the hydrodynamic size distributions of these NP formulations and FIG. 1D shows TEM images that highlight the core size and morphology of each NP formulation. NP synthesis, physiochemical properties for each NP type, the electron diffraction patterns for these NP formulations, and the Fourier transform-infrared spectra of these NP formulations were evaluated as described below.

### ROS Generation in Buffered Aqueous Solution

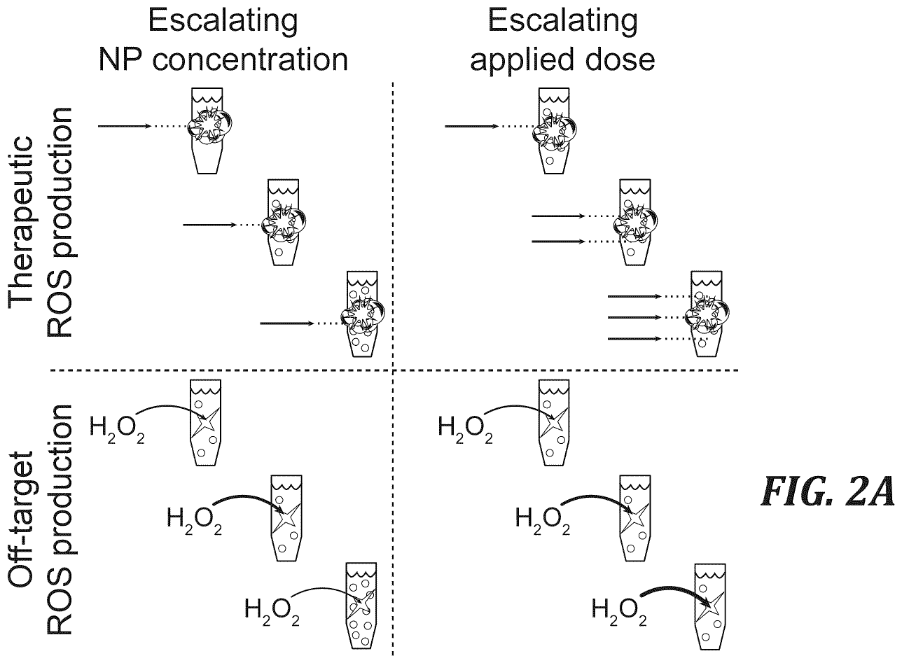

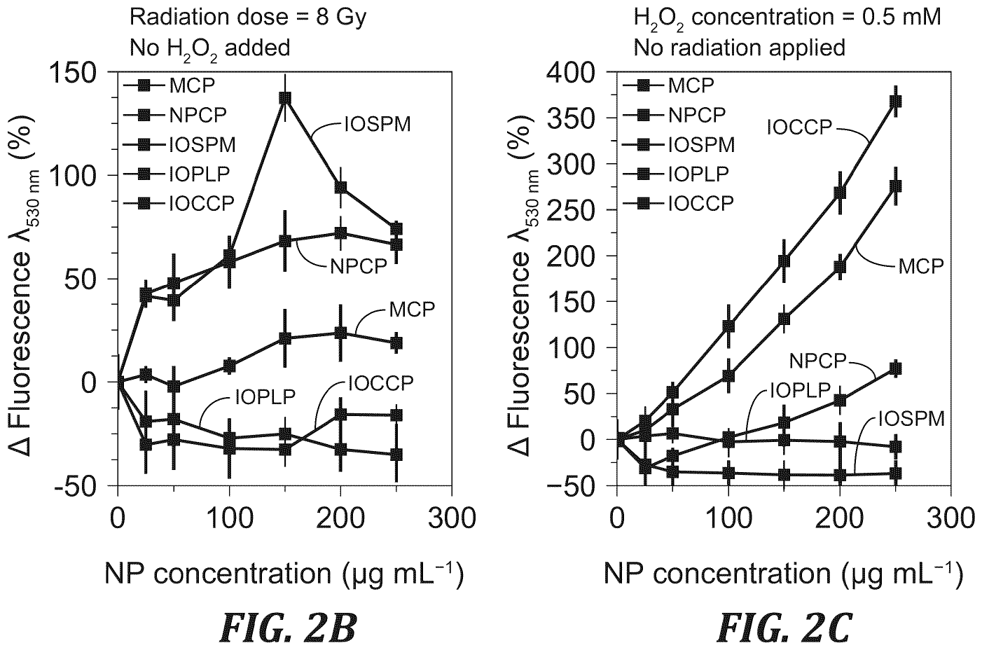

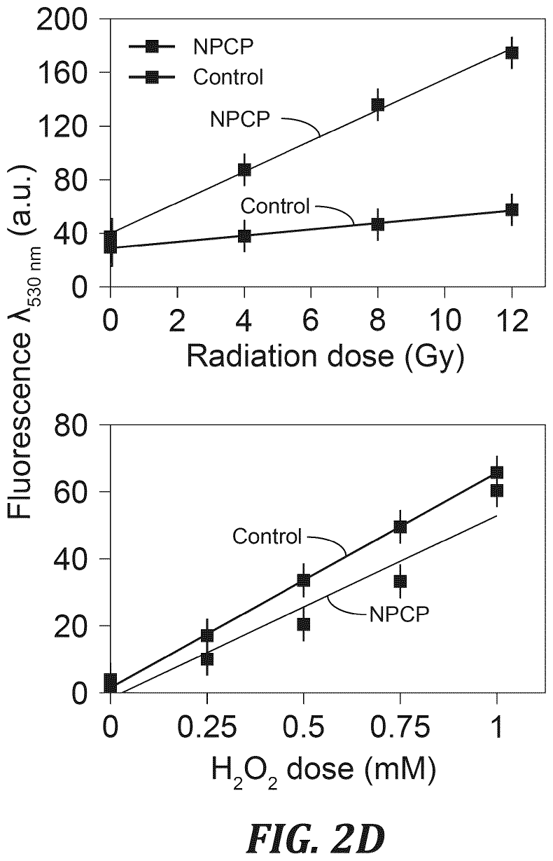

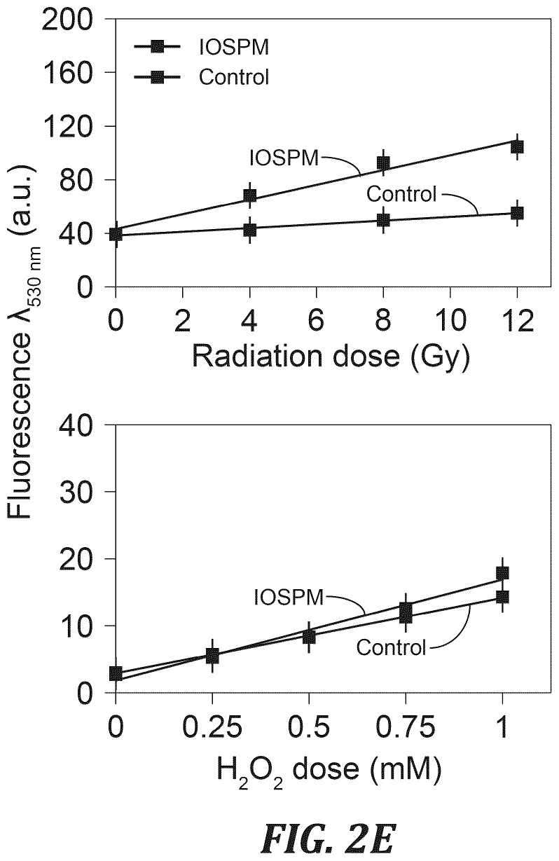

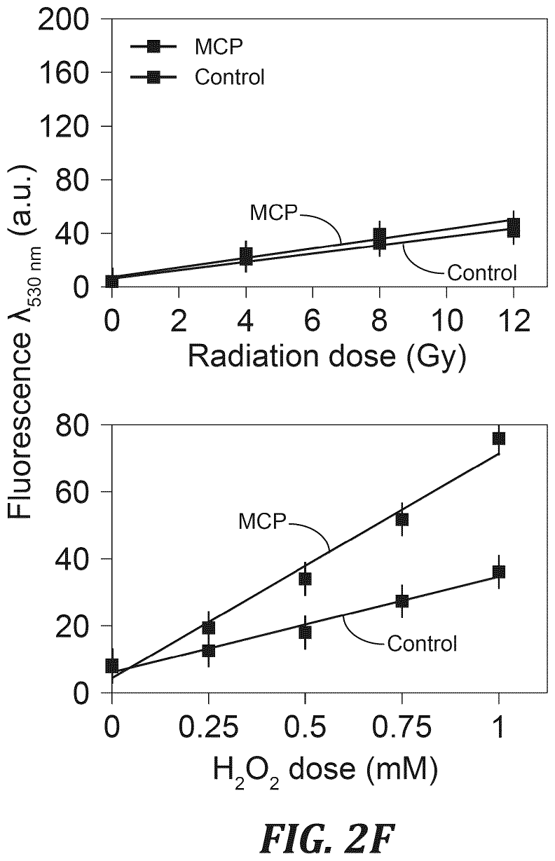

To evaluate the propensity of each NP formulation to generate ROS during radiotherapy, ROS yields were measured in buffered aqueous solution containing NPs and the ROS indicator, 2′,7′-dichloro-dihydro-fluorescein (DCFH). γ-Radiotherapy fluorometric assays were performed, while systematically varying the NP concentration (FIG. 2A). These results were compared to identical experiments, designed with an oxidative chemical stimulus (H₂O₂) substituted for radiation. An ideal NP would generate ROS when exposed to γ radiation, and would not potentiate ROS production under chemical conditions that mimic intracellular oxidation. While ROS production by radiation is the desired therapeutic effect, enhanced ROS with H₂O₂ would allow us to anticipate cytotoxicity of the nanoparticle itself, when not exposed to radiation. The H₂O₂ control also provides reassurance that admixture of the NP with the indicator molecule (i.e., fluorescence quenching) is not responsible for the observed effects.

FIG. 2B shows the result of applying 8 Gy γ-irradiation to 5 different NPs, while varying NP concentration from 0 to 250 μg ml⁻¹. The ROS indicator was present at equal concentration in each reaction, and all experiments were performed in triplicate. The experiment shows that the conversion of DCFH to fluorescent 2′,7′-dichlorofluorescein (DCF) (i.e., ROS production) is greatest when NPCP and IOSPM are present. MCP, identical in shell configuration to NPCP, yields a comparatively low DCFH conversion, only apparent at higher NP concentrations (>150 μg ml⁻¹). Unlike other NP species, IOPLP and IOCCP do not potentiate the conversion of DCFH to DCF in the presence of γ-irradiation, but instead inhibit this conversion at all concentrations, indicating these are not candidate species for biological NMDR. FIG. 2C displays the results of the oxidative control experiment.

FIG. 2C provides data after substitution of 0.5 mM H₂O₂ as the oxidizing stimulus, once again allowing the concentration of each NP species to vary. Within physiologically relevant concentration (<100 μg ml⁻¹), NPCP exhibited little potentiation of oxidative free radical production. IOSPM had a mild ROS-scavenging effect. The enhanced conversion of DCFH to DCF caused by IOCCP and MCP in an oxidizing environment suggests that oxidative damage in vitro or in vivo might occur from these NP systems, which could cause toxicity. The disparity between results of the H₂O₂ experiment (FIG. 2C) and γ-radiation (FIG. 2B) suggests that the T-irradiation results cannot be explained by natural oxidative processes, fluorescence quenching, or some unknown effect from the admixture of NP and DCFH. These results show that the variable of T-irradiation itself is responsible for the potentiation of ROS by NPCP and IOSPM.

A set of additional experiments was performed on NP formulations that yielded ROS potentiation in the presence of T-irradiation as shown above: NPCP, IOSPM, and MCP. The applied radiation dose was varied from 0-12 Gy, while maintaining NP concentration at 25 μg ml⁻¹ (FIG. 2D-2F, top row). With escalating radiation dose, NPCP (FIG. 2D) and IOSPM (FIG. 2E) influenced a conversion of DCFH to fluorescent DCF that was significantly greater than ROS production during concurrent control radiation experiments where NP was absent. The difference in slope between NP and control curves indicates that radiation-derived ROS production is amplified at higher radiation doses. In each case, the difference in control and NP slopes effectively conveys the relative “potency” of the NMDR response for a given particle. As an additional control, each of these experiments was repeated using escalating dose of H₂O₂ to initiate ROS production. Accompanying results in FIG. 2D-2F (bottom row) show that conversion of DCFH to DCF from 25 μg ml⁻¹ NP in oxidizing conditions remains less than, or nearly equivalent to, that produced by H₂O₂ alone, regardless of oxidizing agent concentration. The exception to these results remains MCP. MCP differs from NPCP only in the Fe₂O₃ core composition, and the altered redox state of the maghemite core is associated with a very small difference in slope between NP and no-NP conditions (FIG. 2F, top). In agreement with prior results (FIG. 2C), the action of MCP in non-radiative oxidizing conditions is to generate additional ROS compared to a condition where no particle is present (FIG. 2F, bottom).

The production of ROS by iron oxide NMDR was highly dependent on core composition, capping agent and polymer coating. IOCCP and NPCP were both produced by a co-precipitation synthesis process, have similar core composition, size and morphology, and an outer shell of chitosan-PEG; however, the addition of electron withdrawing catechol groups along the chitosan backbone as a capping agent on IOCCP drastically reduced ROS production induced by T-irradiation. Similarly, IOSPM and IOPLP were both produced by a thermal decomposition synthesis process, share the same core composition, size and morphology, and an outer shell of PEG, yet IOPLP contains an added hydrophobic lipid bilayer between the iron oxide core and outer PEG shell, displacing water from the iron oxide surface. This change in surface configuration of IOPLP may be responsible for the low radiation-induced DCFH conversion rate, as surrounding H₂O is excluded from the region adjacent to the NP core.

The results do not show a clear correlation between core size or hydrodynamic size and NMDR efficiency, although evaluation of NPs with a greater range of core and hydrodynamic sizes may be required to reveal a true trend. These results highlight a missing link in the understanding of the mechanism of high-Z-species-mediated ROS production. The choice of capping agent, polymer coating, and core composition can yield unique surface energy and bond structure along the metal-polymer interface of NPs. It is well known that perturbation of these parameters leads to great differences in magnetic properties of iron oxide NPs due to changes in electronic configuration of atoms at the iron oxide surface. In this case, such structural changes can impact the communication of Auger photoelectrons to surrounding water molecules and ROS-indicator species.

Based on the composite data from ROS generation in buffered aqueous solution, it was concluded that a magnetite core is essential to achieving NMDR effects from an iron oxide NP, and the incorporation of maghemite is counterproductive. NPCP proved to have the greatest relative increase in ROS production for a given increase in T-radiation dose. Given the known biocompatibility and blood-brain barrier (BBB) transport properties of NPCP, it was chosen as the represented system described herein.

### In Vitro Iron Oxide NP-Mediated Deposition of Radiation

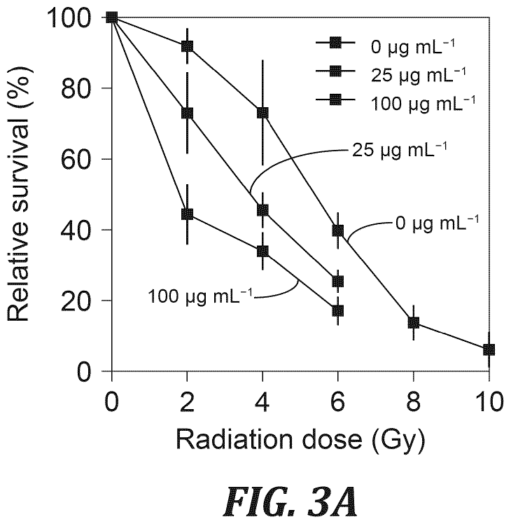

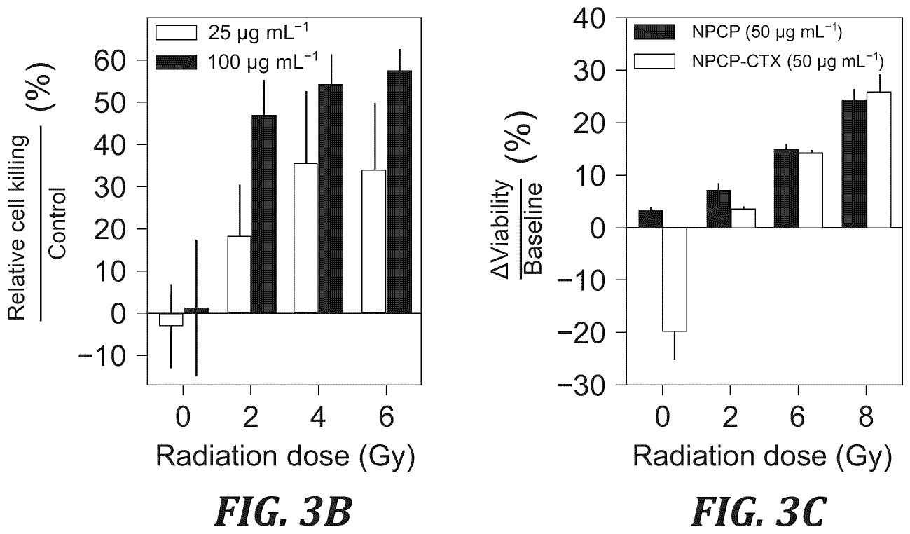

The NMDR effect of NPCP was tested on GBM cells in vitro, using assays of proliferative survival and metabolic activity. FIG. 3A shows the effect of NMDR on relative clonogenic survival in the SF767 GBM cell line; cells were incubated in solution containing NPCP for 12 hours at two separate doses. Cells then received variable dose γ-radiation. The plating efficiencies of NPCP-treated and NPCP-untreated cells were similar indicating that NPCP alone did not induce a measurable effect on clonogenic survival. The amount of iron oxide loaded into cells at the two doses was assessed using an iron quantification assay and was shown to be roughly double at treatments of 100 μg ml⁻¹ compared to 25 μg ml¹. The fractional decrease in tumor cell survival roughly doubled with treatments of 100 μg ml⁻¹ compared to 25 μg ml⁻¹ (FIG. 3B) for all radiation doses. The proportion of tumor cells killed with combined NP/radiation, compared to radiation alone, increased with escalating radiation dose, from 18±16% (2 Gy) to 34±16% (6 Gy) using 25 μg ml⁻¹ NPCP and from 47±10% (2 Gy) to 57±9% (6 Gy) using 100 μg ml⁻¹ NPCP.

To support the findings from clonogenic assay, an alamar blue assay for tumor cell metabolic activity and viability was performed using GBM6 cells exposed to variable doses of γ-radiation (FIG. 3C). Unlike the clonogenic assay, alamar blue did not require cell replication in culture after treatment. GBM6 cells were loaded for 12 hours with 50 μg ml⁻¹ NPCP, followed by γ-radiation and subsequent incubation for an additional 3 days. The relative decrease in viability after combined radiation/NPCP compared to radiation alone ranged from −7.1±1.3% after 2 Gy/NPCP to −24±2.3% after 8 Gy/NPCP. The viability of non-radiated cells incubated with NPCP was similar to that of entirely untreated cells (3.4±0.1% change). It has been previously shown that conjugating the tumor-targeting peptide chlorotoxin (CTX) to NPCP promotes NP accumulation in the tumor site; thus, to enhance the delivery of NPCP to orthotopic GBM tumors, CTX was conjugated to NPCP (NPCP-CTX). TEM images showed the sizes of NPCP and NPCP-CTX and no apparent size difference between the two nanoparticle formulations was observed. The hydrodynamic diameters of NPCP and NPCP-CTX were measured over a time period of 7 days to assess the NP size stability, which shows a minimal hydrodynamic size increase for both NPCP and NPCP-CTX over this period. A direct comparison of CTX-conjugated and non-conjugated NPCP was performed in FIG. 3C to ensure that effects of CTX on the tumor cell (e.g., radiosensitization) do not account for any potential therapeutic results observed in vivo. FIG. 3C includes a comparison of data from NPCP-CTX at each radiation dose. The relative change in cell viability was roughly similar for NPCP and NPCP-CTX at radiation doses of 6 and 8 Gy indicating that the conjugation of CTX to NPCP did not significantly alter the NMDR efficacy of NPCP. Radiosensitization by CTX is therefore not expected to influence in vivo results.

To show improved cell targeting and uptake of NPCP by tumors when NPCP is conjugated to CTX, NPCP was synthesized and then conjugated to a fluorophore, Cy5, to generate NPCP-Cy5. The NPCP-Cy5 was divided into two groups: a group conjugated to CTX NPCP-Cy5-CTX and a group left unmodified (NPCP-SF763 human GBM cells were incubated with varying concentrations of either NPCP-Cy5 or NPCP-Cy5-CTX (10, 25, and 50 μg ml⁻¹). After 12-h incubation, the cell colonies were imaged by fluorescence microscopy to determine the degree of cell uptake of NPs. Cells incubated with NPCP-Cy5-CTX exhibit a much higher fluorescence intensity than cells incubated with NPCP-Cy5, suggesting that the conjugation of CTX to NPCP promotes GBM cellular uptake of the NPs.

### In Vivo Iron Oxide NP-Mediated Deposition of Radiation

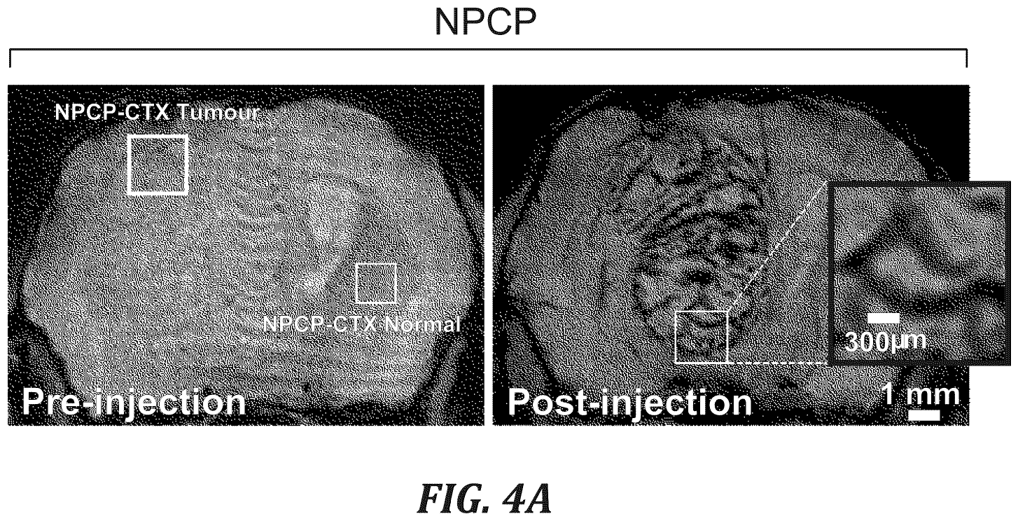

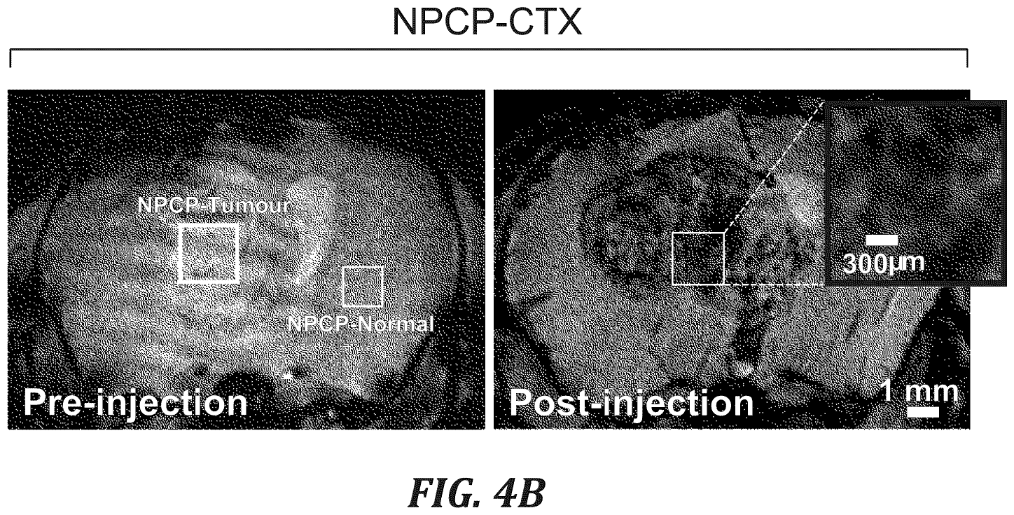

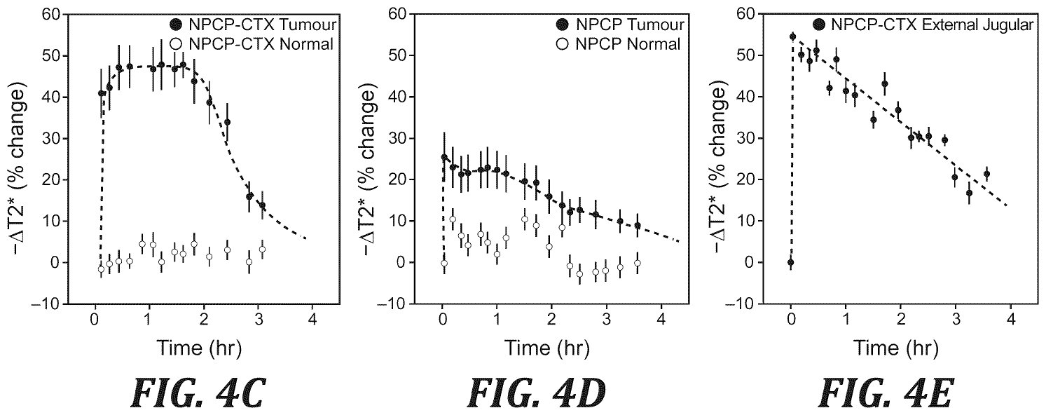

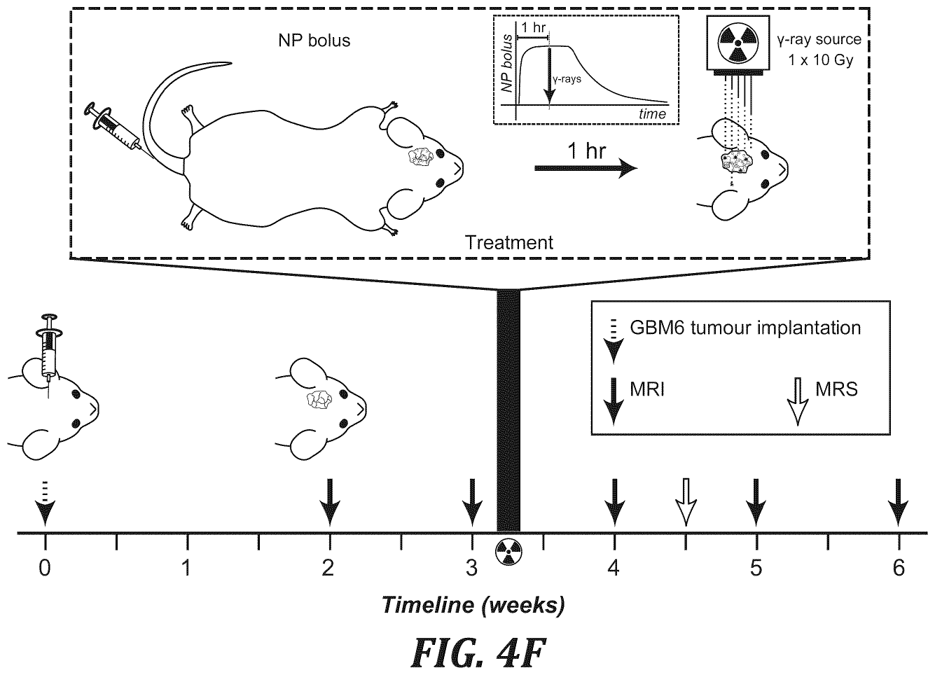

The superparamagnetic behavior of iron oxide NPCP allowed visualization of particles via MRI in vivo immediately after injection. Tracking of the NPs assists in selecting an acceptable time for delivery of γ-radiation after NP injection. The transverse relaxivity of NPCP and NPCP-CTX was 40.0±0.8 mM⁻¹ s⁻¹ and 40.3±1.0 mM⁻¹ s⁻¹, respectively. Sequential T₂*-weighted imaging was performed before and after tail vein injection of NPCP-CTX and NPCP at a total body dose of 8.5 mg kg-1 iron oxide. Representative T₂*-weighted images displaying orthotopic primary GBM6 xenograft tumors 1 hour after injection are shown for NPCP-CTX (FIG. 4A) and NPCP (FIG. 4B), with relative signal change in tumor and contralateral brain parenchyma shown in FIGS. 4C and 4D for NPCP-CTX and NPCP, respectively. Greater T₂*-weighted signal change is observed in tumor compared to normal brain tissue, and a greater signal change is observed using NPCP-CTX rather than NPCP. Prior investigation has suggested that CTX-conjugation yields improved NPCP uptake past the BBB. The MRI technique does not discriminate between intravascular and extravascular NP. Relative T₂*-weighted signal change within the blood was determined by monitoring the external jugular vein and is shown in FIG. 4E. A linear clearance profile of NPCP-CTX from the blood was observed. A plateau appears in tumor tissue before NP elimination; such a difference in the pharmacokinetics of NPCP-CTX between blood and tumor tissue indicates the extravasation of NPCP-CTX from the blood into the tumor region at which point NPCP-CTX is retained for a period before washout occurs. Although prior research has suggested that intracellular NPs may persist for 5 days, γ-radiation was delivered 1 hour after tail vein injection, when the greatest concentration of overall NPs was present in the bulk tumor as dictated by time-resolved MR imaging. FIG. 4F illustrates the planned course of treatment and monitoring. MRI of mice was performed weekly, and magnetic resonance spectroscopy (MRS) was obtained 10 days after treatment. The treatment scheme involving a single NP bolus injection and single radiation dose was selected as a proof-of-principle demonstration for the combined therapy.

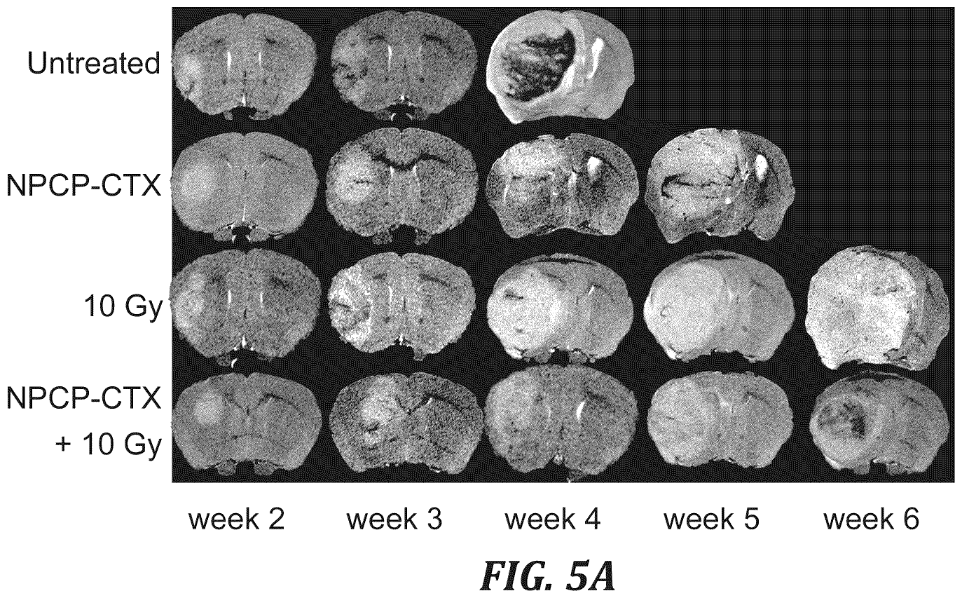

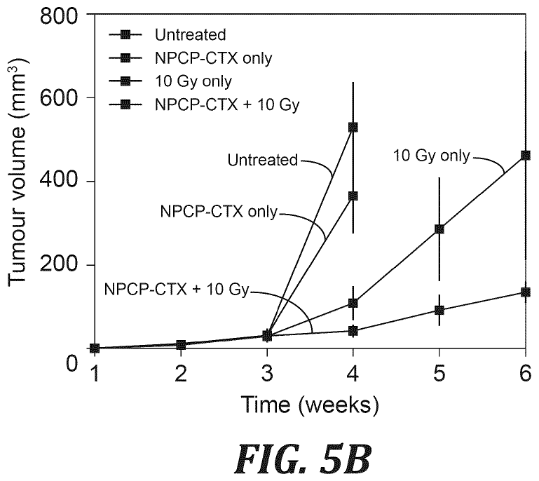

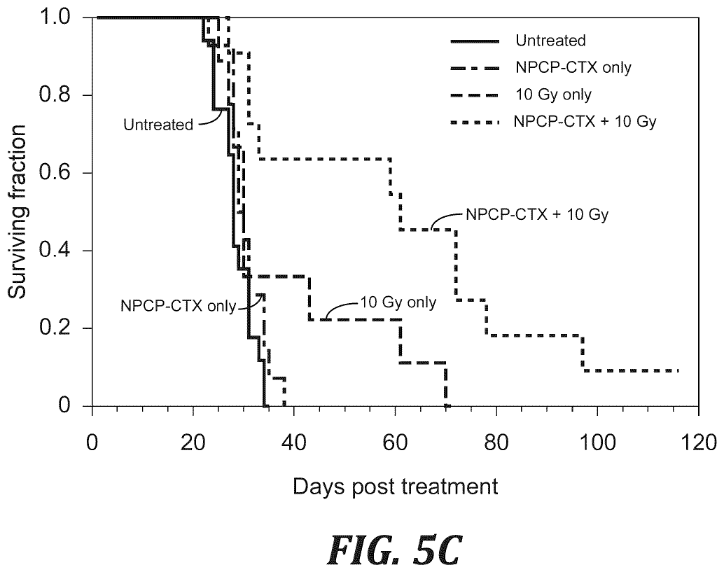

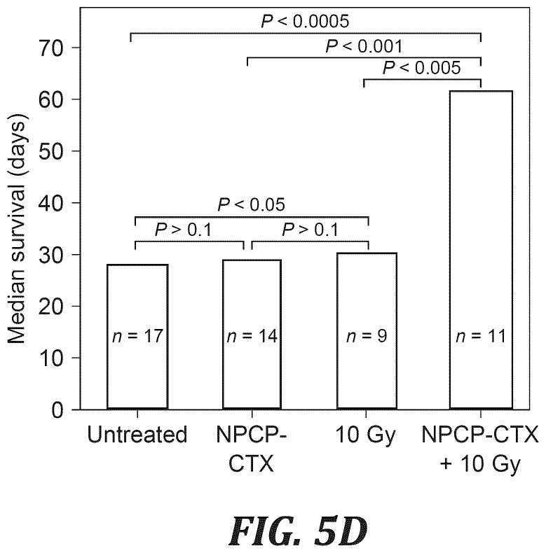

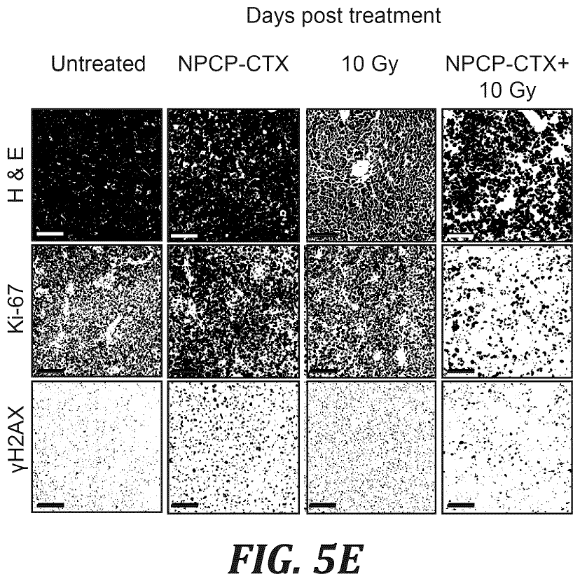

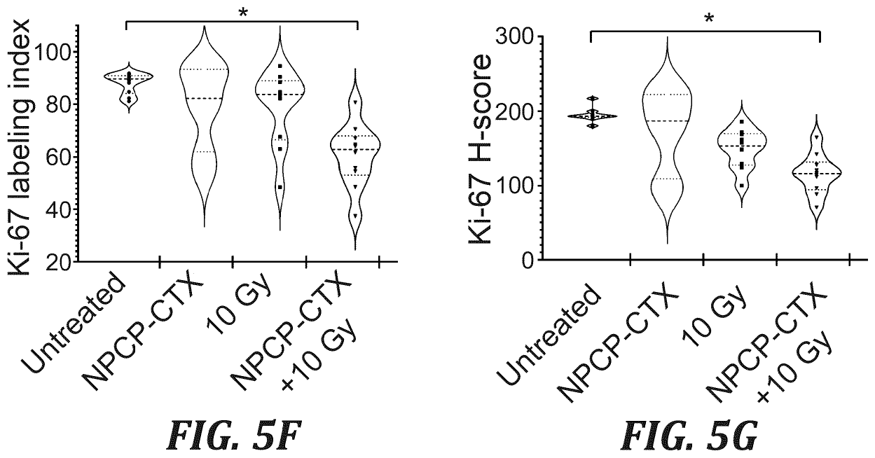

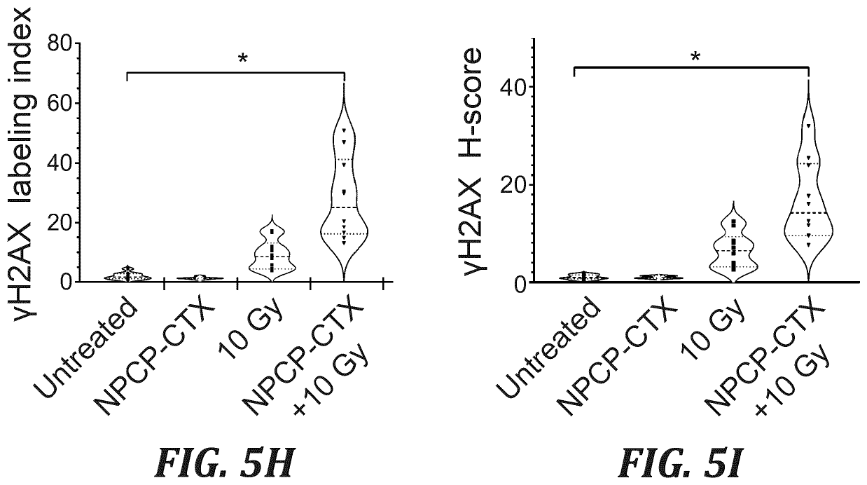

NPCP-CTX with concurrent 10 Gy single dose γ-radiation was delivered to mice bearing orthotopic GBM6 xenograft tumors, with examination of tumor size and survival. Control groups included untreated mice, mice receiving only NPCP-CTX, and mice receiving only γ-irradiation. 3 mice in each group were designated to serial MRI study, receiving weekly scans. Representative T₂-weighted images of tumor development in each of the experimental categories are provided in FIG. 5A. Average volumetric tumor size is displayed in FIG. 5B. Tumor growth profiles of the untreated and NPCP-CTX-treated groups were not significantly different. Treatment with a single 10 Gy dose of γ-irradiation showed an expected reduction in tumor growth rate. Linear regression analysis performed on the 10 Gy only and NPCP-CTX+10 Gy tumor growth curves showed that the NPCP-CTX+10 Gy group exhibited a 3-fold decrease in tumor growth rate compared to the 10 Gy only group from weeks 4 through 6. Kaplan-Meier survival curves are shown in FIG. 5C, with log rank statistical analysis of median survival supplied in FIG. 5D. Animals treated with NPCP-CTX concomitantly with a 10 Gy dose of γ-irradiation showed a 2-fold increase in median survival as compared to animals receiving only γ-irradiation, with a median survival time of 61 days in comparison to 30 days for γ-irradiation alone, 29 days for NPCP-CTX control and 28 days for untreated animals. The log rank statistic for comparison between the NPCP-CTX+10 Gy group and the 10 Gy-only group showed a statistically significant difference (P=0.003). These results suggest that the combined effect of targeted NPCP-CTX and γ-irradiation confers a survival advantage to mice bearing GBM6 brain tumors. Pathological and immunohistochemical (IHC) tumor tissue analysis was performed on separate mice sacrificed 10 days after treatment administration (FIGS. 5E-5I). Fractional Ki-67 positivity within the tumor, a marker of cellular proliferation, was calculated via standard labeling index (FIG. 5F) and weighted H-score (FIG. 5G) methods, demonstrating a reduction of overall Ki-67 positivity in the combined NPCP-CTX+10 Gy treatment group compared to controls. Fractional γH2AX was calculated in a similar manner (FIGS. 5H and 5I), in each case showing an increase in tumor γH2AX positivity for the NPCP-CTX+10 Gy treatment group compared to controls, suggesting an increase in DNA damage as a result of the combined treatment method. As the effects of ionizing radiation are known to be mediated through DNA damage, as well as secondary sequella including ROS-induced lipid oxidation, the increase of γH2AX positivity correlates well with the expected mode of action for Auger therapy. γH2AX was assessed over 10 random high-powered areas in the adjacent brain tissue from each of the four groups. γH2AX positive cells in adjacent brain tissue were lower in the NPCP-CTX +10 Gy treated group as compared to the group receiving radiation only, which provides additional support that the combined therapy cause little to no damage to the adjacent brain tissue.

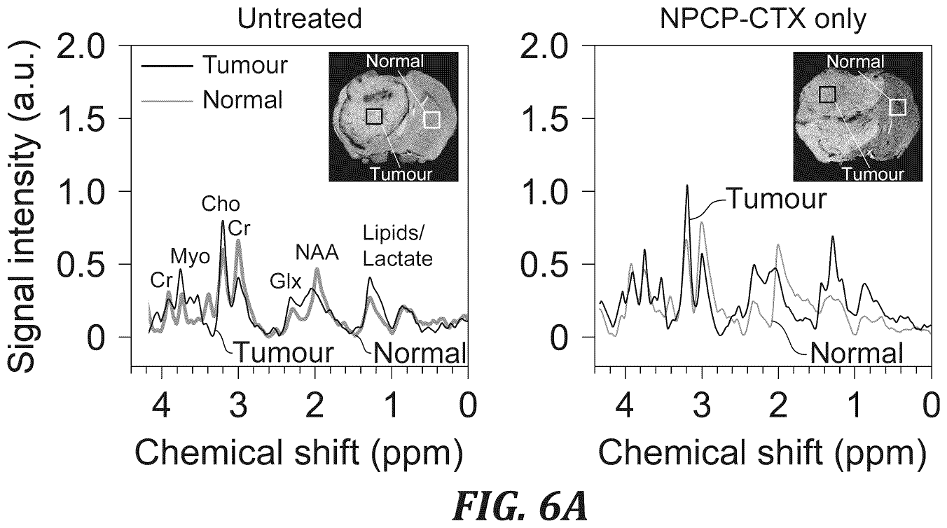

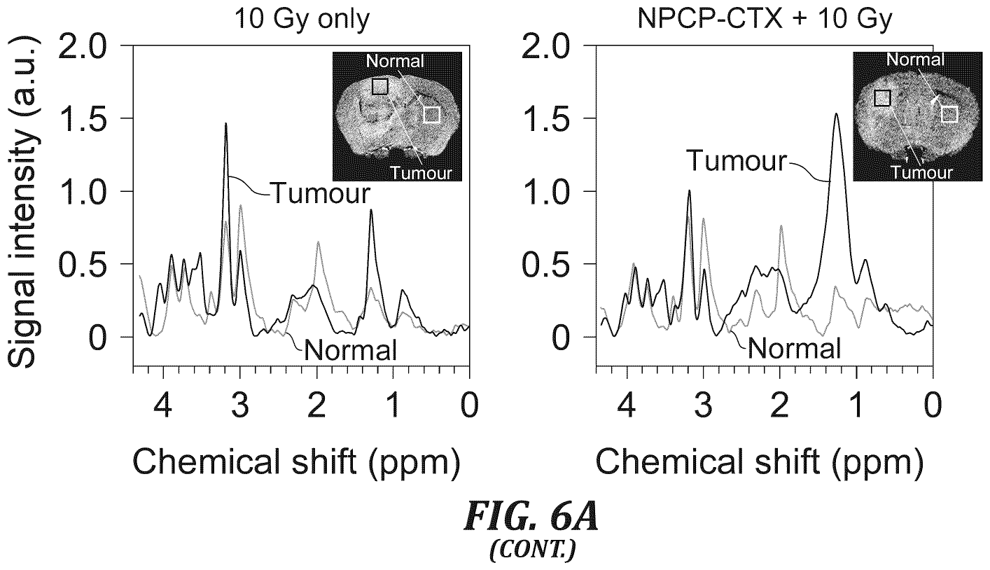

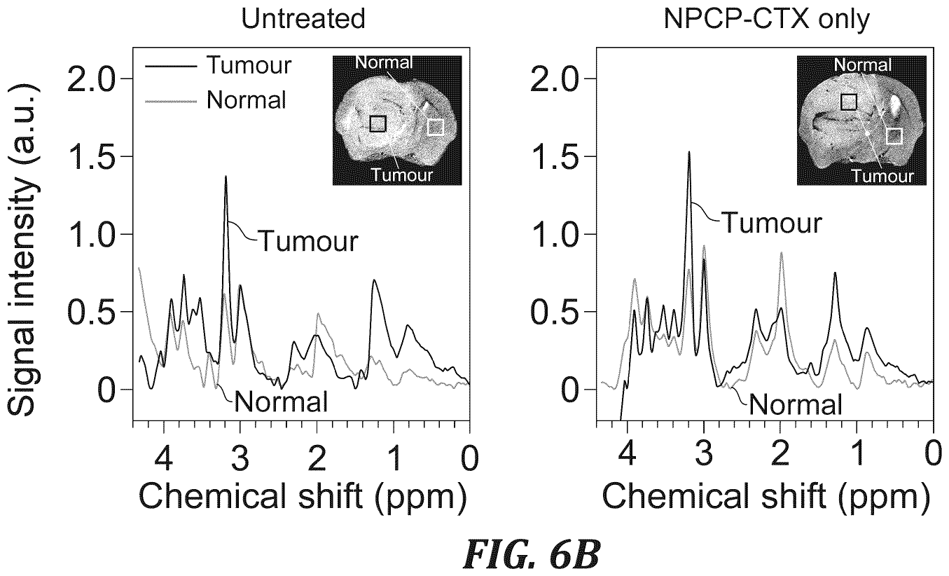

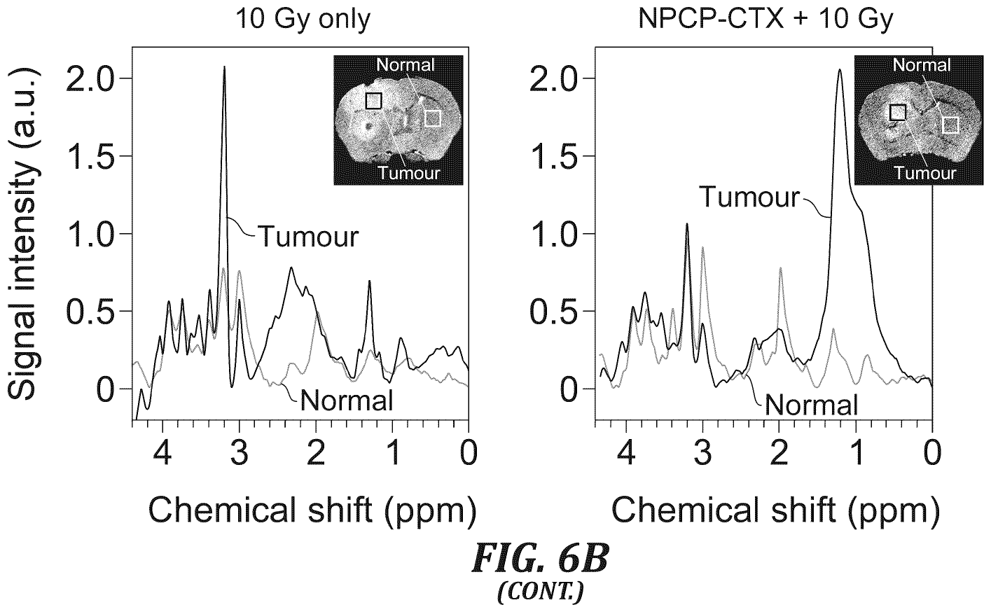

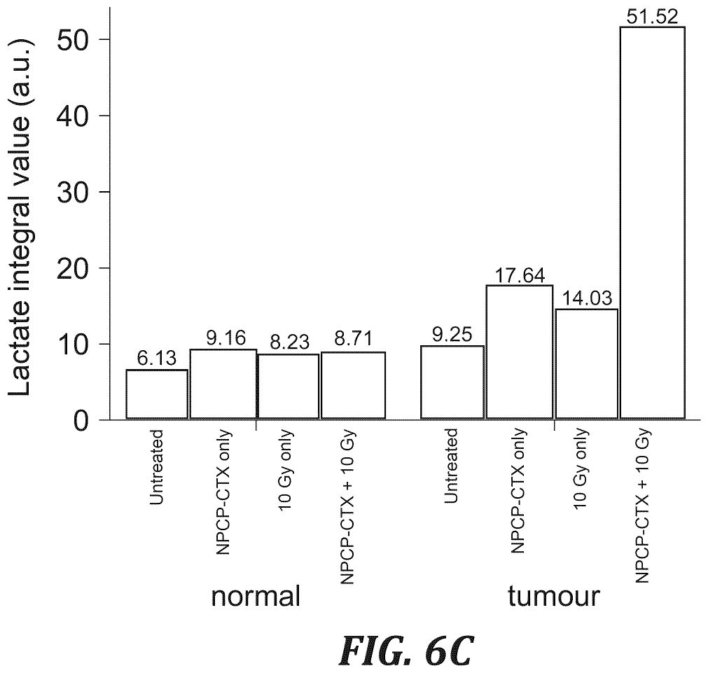

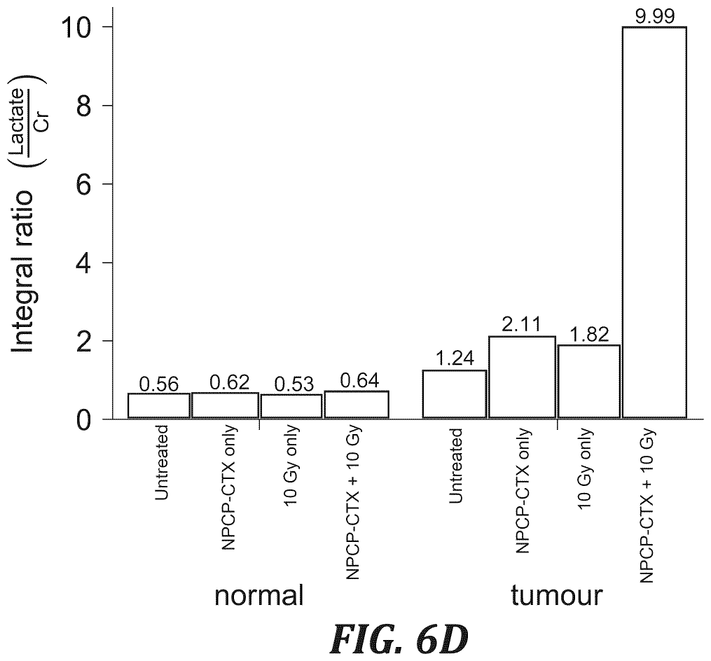

In vivo MRS was performed as a useful adjunct to IHC, to reveal potential in vivo functional neurochemical effects of our treatment modality. Distinct benefits of MRS include (i) the examination of living tissue, (ii) the ability to observe results that may only be evident shortly after treatment and while mice remain alive for survival studies, and (iii) testing of animals at an equivalent time point. MRS data were collected from 2 mice from each of the conditions (8 mice total), 10 days after treatment. This intermediate time delay was short enough to ensure that at least 2 surviving mice were present in each imaging group and was long enough to allow elimination of NP accumulated in tumor tissue, which would otherwise generate imaging artifact that would prevent collection of data. Furthermore, the time delay provided results that measured a durable effect on tumor and adjacent brain tissue, rather than a transient effect that would only be observed immediately after treatment. FIG. 6A displays spectra measured for one mouse in each cohort and FIG. 6B shows the spectra from a second cohort. Chemical shifts for metabolites are designated on the first spectrum, including creatine (Cr), myoinositol (Myo), choline (Cho), glutamate and glutamine (Glx), N-acetylaspartate (NAA) and lipid/lactate. MRS data demonstrate a marked increase in concentration of lactate in tumor tissue of the NPCP-CTX+10 Gy group compared to all other treatment groups. The sharp spike in lactate signifies necrosis and ischemia of tumor tissue. The chosen regions of interest (ROI) for MRS are displayed on accompanying images, and in all cases consisted of homogeneous tumor tissue and adjacent brain, avoiding placement of the ROI over any obvious region of tumor necrosis. The results suggest an increase in local damage caused by radiation+NPCP-CTX delivered to the tumor, an effect which is not observed in adjacent brain despite exposure to the same treatment. Because all tumors were formed from the same group of GBM6 cells, these results cannot be explained by a difference in tumor type, nor can they be attributed to spatially-selective radiation delivery as whole-brain radiation was used. A reasonable conclusion is suggested, that enhanced tumor tissue damage only occurs in the region where NPs are preferentially targeted by CTX. Other metabolites identified in the spectra remained relatively constant across all treatment groups. To quantify the increase in lipid/lactate for the NMDR treatment group relative to other groups, the average integral of the lactate peak was calculated for each spectrum (FIG. 6C). Furthermore, the ratio of the lactate to Cr peak was computed as a reference for peak normalization (FIG. 6D), since Cr is relatively stable in the brain and is thus used for calculating metabolite ratios. As expected for neoplastic tissue, the Cho:Cr ratio is greater than 1:1 for all tumor spectra, and less than 1:1 for all contralateral brain tissue spectra, with NAA peaks that are greater in normal brain than those in tumor.

A significant benefit of targeted NMDR therapy is the ability of NPs to home to cellular metastases and alter the characteristics of radiation deposition at these imaging-occult sites of concern.

The present disclosure provides an innovation on the classic radiotherapy model using biocompatible targeted iron oxide NPs to facilitate production of Auger photoelectrons. Chemical experiments were performed on a suite of iron oxide NPs, and demonstrated a dependence of radiation-induced ROS production on the redox state of the iron oxide core, the NP capping agent, and the polymer coating. Magnetite particles produced a high NMDR effect while maghemite particles did not; the inclusion of electron withdrawing catechol groups as a capping agent diminished ROS yield; the sequestration of water from the magnetite core by incorporation of a lipid layer significantly reduced ROS production. Furthermore, NPs synthesized with a maghemite core or an electron-withdrawing catechol capping agent led to undesirable production of ROS under oxidizing conditions. After identifying a highly functional and biocompatible NP formulation (NPCP), this resulting particle was tested in culture with two GBM cell lines (SF767 and GBM6). The combined γ-radiation+NPCP treatment was found to yield enhanced cell killing compared to radiation or NP delivery alone. NPCP was conjugated to the tumor-targeting peptide CTX, which has been demonstrated to be biocompatible and has the ability to cross the BBB and accumulate in GBM tumor. While CTX was expected to increase the extent of in vivo BBB NP penetration, no additional cytotoxicity was observed in cell culture by comparison of NPCP and NPCP-CTX. Using time-resolved T2*-weighted MRI, the optimal timing was determined for delivery of NPCP-CTX and γ-radiation in tandem in mice bearing GBM6 tumors. Treatment with NPCP-CTX combined with γ-radiation was performed in vivo. In a late stage, rapidly lethal GBM6 tumor, a 2-fold improvement in survival and 3-fold suppression in tumor growth rates was shown, compared to mice receiving radiation alone. These results are supplemented by histology/IHC performed 10 days after treatment in separate mice, and in vivo MRS 10 days after therapy performed on mice from the primary experimental groups. Spectra reveal that the combined therapy generates a large increase in lactate within tumor, that is not observed in normal brain.

The present disclosure provides a tumor-targeted iron oxide NP formulation for intracranial NMDR having demonstrated effectiveness in a human-derived orthotopic malignant cancer model. The effect of NP design parameters, including core redox state, iron oxide surface ligand, and polymer coating on reactive oxygen species (ROS) production, were evaluated. Representative iron oxide nanoparticles were tested in iron oxide NMDR against GBM6, a cell line derived from human GBM and serially passaged in vivo, retaining its clinically relevant phenotype. Late-stage GBM scenario was simulated by waiting 3 weeks post-inoculation, allowing tumor volumes to reach 5% of total brain volume before applying treatment regimens. Mice bearing GBM xenografts that received simultaneous tumor-targeted iron oxide NP and γ-irradiation demonstrated a slowed tumor growth rate and significantly prolonged median survival compared to mice treated only with either γ-irradiation or NPs. The present disclosure indicates that tumor-targeted iron oxide NMDR is an appealing therapeutic strategy.

As used herein, the term “about” refers to ±5% of the specified value.

### EXPERIMENTAL

### Materials

All chemicals were purchased from Sigma-Aldrich (St. Louis, MO) unless otherwise stated. Cell culture reagents including Dulbecco's Modified Eagle Medium (DMEM) and antibiotic-antimycotic were purchased from Invitrogen (Carlsbad, CA). Fetal bovine serum (FBS) was purchased from Atlanta Biologicals (Lawrenceville, GA). 3-(Triethoxysilyl)propylsuccinic anhydride (SATES) was purchased from Gelest (Arlington, VA).

### Nanoparticle Synthesis

Maghemite NP. Uncoated iron oxide NPs were synthesized via co-precipitation, mixing 570 mg of Fe³⁺ iron chloride in 18 ml of degassed deionized (DI) water. This solution was then passed through a 0.2 μm cellulose acetate filter. Next, the solution was placed in a sonicated water bath heated to 40° C. Ammonium hydroxide (14.5 M) was slowly titrated into the solution over a period of 45 min until a final pH of 10.5 was reached, ensuring complete NP nucleation.

NPCP and NPCP-CTX. Iron oxide NPs were synthesized and coated with poly(ethylene glycol) (PEG) grafted onto depolymerized chitosan (NPCP) by a co-precipitation method described in F. M. Kievit, O. Veiseh, N. Bhattarai, C. Fang, J. W. Gunn, D. Lee, R. G. Ellenbogen, J. M. Olson, M. Zhang, Adv. Funct. Mater. Mater. 19 (2009) 2244-2251 and Z. R. Stephen, F. M. Kievit, O. Veiseh, P. A. Chiarelli, C. Fang, K. Wang, S. J. Hatzinger, R. G. Ellenbogen, J. R. Silber, M. Zhang, ACS Nano 8 (2014) 10383-10395. Chlorotoxin (CTX, Alamone Laboratories, Jerusalem, Israel) was conjugated to NPCPs (NPCP-CTX) as described in F. M. Kievit, O. Veiseh, C. Fang, N. Bhattarai, D. Lee, R. G. Ellenbogen, M. Zhang, ACS Nano 4 (2010) 4587-4594.

IOSPM. Oleic acid coated iron oxide NPs (IOOA) were synthesized as described in J. Park, K. An, Y. Hwang, J.-G. Park, H.-J. Noh, J.-Y. Kim, J.-H. Park, N.-M. Hwang, T. Hyeon, Nat. Mater. 3 (2004) 891-895. To confer water solubility, a ligand exchange process was used to provide a hydrophilic PEG coating. 50 mg of IOOA was suspended in 43 ml of anhydrous toluene followed by addition of 50 μl of triethylamine in a 3-neck round-bottom flask fitted with a Graham condenser. The flask was sealed with rubber septa and purged with nitrogen. The solution was heated to 100° C. and 0.10 ml of SATES was added to the flask. 187.5 mg of mPEG2K—NH₂ was dissolved in 7 ml of anhydrous toluene and the resultant solution was added to the flask 15 min after the addition of SATES. An additional 50 μl of SATES was injected 1 hour after the mPEG2K—NH₂ injection, and the solution was reacted for a further 6 hours and 45 min. The solution was transferred to a single neck round-bottom flask and NPs were precipitated with hexane. The NP precipitate was dispersed in tetrahydrofuran (THF), sonicated for 10 min and precipitated with hexane. The resulting NP pellet was suspended in 10 ml anhydrous THF and sonicated for 10 min. 62.5 mg of mPEG2K—NH₂ and 187.5 mg of 2000 MW bis(amine) functionalized PEG (PEG2K-bis(amine)) was dissolved in 12 ml of anhydrous THF and added to the NP solution. The flask was then sealed with a septum and purged with nitrogen. 12.5 mg of N,N-dicyclohexylcarbodiimide (DCC) was dissolved in 2 ml of anhydrous THF and added to the flask, and the reaction solution was placed in a sonication bath at 25° C. and allowed to react for 16 hours. Fully PEGylated NPs were precipitated with hexane, dispersed in 20 ml ethanol, sonicated for 10 min and precipitated again with hexane. The pellet was fully dried and dispersed in PBS with sonication for 10 min.

IOPLP. IOOA (1 mg) was mixed with 1 ml of acetone and sonicated for 10 min to remove free oleic acid. The mixture was placed on a magnet to separate IOOA from the solution. Acetone was decanted and IOOA was dried with nitrogen flow. IOOA and 20 mg of 18:0 PEG2000 PE (Avanti Polar Lipids, Inc., Alabaster, Al) was dispersed in 1 ml chloroform. 4 ml of DMSO was added drop wise with constant stirring, followed by rocking at room temperature for 30 min. Chloroform was removed by vacuum and 16 ml of DI water was added drop wise with constant stirring. The mixture was then concentrated and purified using an Amicon Ultra centrifugal filter (EMD Millipore, Billerica, MA) following the manufacturer's instructions. Once concentrated to 1 ml, the mixture was washed with 2 ml of DI water three times to remove residual DMSO.

IOCCP. Iron oxide NPs coated with a catechol modified chitosan-PEG copolymer (IOCCP) were synthesized as described in Z. R. Stephen, C. J. Dayringer, J. J. Lim, R. A. Revia, M. V Halbert, M. Jeon, A. Bakthavatsalam, R. G. Ellenbogen, M. Zhang, ACS Appl. Mater. Interfaces 8 (2016) 6320-6328.

### Nanoparticle Characterization

The hydrodynamic size of NPs was determined by dynamic light scattering at 100 g ml⁻¹ in 20 mM HEPES buffer (pH 7.4) using a Zetasizer Nano (Malvern Instruments, Worcestershire, UK). Transmission electron microscopy (TEM) images were acquired with an FEI TECNAI F20 TEM (Hillsboro, OR) operating at 200 kV.

### Radiation Delivery

Cells in culture and tumor-bearing animals were exposed to ionizing radiation at room temperature using a calibrated Mark I ¹³⁷Cs γ-irradiator (J. L. Shepherd and Associates, Glendale, CA). The dose rate for irradiation was 1.1 Gy min⁻¹. For in vivo experiments, the anesthetized mouse was positioned below an overhead radiation source, with the region from caudal skull base to the feet surrounded with continuous 50 mm thickness lead, to shield off-target radiation.

### ROS Measurement

Evaluation of ROS production by NPs in aqueous solution were performed using the 2′,7′-dichloro-dihydro-fluorescein diacetate (DCFH-DA) assay. Appropriate NP volumes were added to PBS buffer, resulting in final volumes of 200 μl held in 96 well-plates. All experimental mixtures were prepared in dark conditions to prevent autoconversion of DCFH-DA by ambient light. Each well was loaded with 100 μM DCFH-DA. For ROS production, either ¹³⁷Cs-γ-rays or H₂O₂ was delivered at the stated dose. For H₂O₂ experiments, equal volume was maintained across experimental and control solutions by addition of either H₂O₂ or PBS, respectively. ROS generation was monitored by conversion of non-fluorescent DCFH into fluorescent DCF (SpectraMax i3 microplate reader; Molecular Devices, Sunnyvale, CA). Excitation and emission wavelengths were 480 nm and 530 nm, respectively.

### Cell Culture

Human GBM cells from the SF767 line were obtained from the tissue bank of the Brain Tumor Research Center (University of California-San Francisco, San Francisco, CA) and maintained in DMEM supplemented with 10% FBS and 1% antibiotic-antimycotic. GBM6 cells were obtained from Mayo Clinic and maintained as flank tumors in nude mice. Cultures were held at 37° C. in a humidified incubator with 5% CO₂ (B. L. Carlson, J. L. Pokorny, M. A. Schroeder, J. N. Sarkaria, Curr. Protoc. Pharmacol. 52 (2011) 1-14).

### Clonogenic Survival Assay

SF767 cells were assessed via clonogenic assay for proliferative survival, after incubation with variable NPCP concentration and delivery of variable radiation dose. Full details of clonogenic method as described in Z. R. Stephen, F. M. Kievit, O. Veiseh, P. A. Chiarelli, C. Fang, K. Wang, S. J. Hatzinger, R. G. Ellenbogen, J. R. Silber, M. Zhang, ACS Nano 8 (2014) 10383-10395. Briefly, NPCP concentrations of 0, 25 and 100 μg Fe ml⁻¹ in DMEM were added to 6-well plates containing 250 cells/well. Cells were incubated for 12 hours at 37° C. in air containing 5% CO₂, followed by 2× washing with PBS, and returned to DMEM. Plates were then exposed to variable γ-irradiation doses of 0, 2, 4, 6, 8, and 10 Gy. Incubation was resumed for 8 days, and during this time, colonies were counted by light microscopy, and only colonies with 50 or more cells were counted. Survival (mean±s.d.) is expressed as the ratio of colonies formed by treated cells to that of untreated cells (i.e., no γ-irradiation). All clonogenic survival studies were performed in sextuplicate. Plating efficiency was determined at all NP incubation concentrations to ensure that NP delivery did not alter the number of viable cells. Separate culture wells were used for cell ferrozine assay and performed in triplicate to determine the amount of intracellular iron resulting from NP incubation at variable concentration.

### Alamar Blue Assay

GBM6 cells were incubated for 12 hours on 12-well culture plates at 37° C. in a 50 g ml⁻¹ solution of NPCP or NPCP-CTX. After 2×washing with PBS, cells were counted and vials of equivalent cell number were exposed to γ-irradiation doses of 0, 2, 6, or 8 Gy. Cultures were allowed to incubate after treatment for 3 additional days. Cells were detached and resuspended in 1 ml of DMEM containing 10% alamar blue reagent (resasurin resazurin). After 90 min additional incubation, 300 μg of supernatant was removed and added to black 96-well microplate designed for fluorescence assay. Resazurin detects cell metabolic by converting from a nonfluorescent dye to the highly red fluorescent dye resorufin in response to chemical reduction of growth medium resulting from cell growth. Conversion of resazurin to resorufin was measured using excitation and emission wavelengths of 560 nm and 590 nm, respectively. The fluorescent signal is proportional to the number of living cells in a sample.

### In Vivo Survival Study

All animal studies were conducted in accordance with approved protocols from the University of Washington's Institute of Animal Care and Use Committee (IACUC) and federal guidelines. Female, nude athymic mice were used in all animal studies. Human-derived GBM6 cells were thawed and maintained through 2 passage cycles as a flank tumor, prior to sterile intracranial implantation as described in Z. R. Stephen, F. M. Kievit, O. Veiseh, P. A. Chiarelli, C. Fang, K. Wang, S. J. Hatzinger, R. G. Ellenbogen, J. R. Silber, M. Zhang, ACS Nano 8 (2014) 10383-10395. Tumor growth was monitored with MRI for 12 mice, with 23 days between implantation and treatment, allowing for tumors to grow to approximately 30 mm³ in size. At the third week, tumors were reliably visible in all 12 imaged mice. Mice were randomized to groups for no treatment, NP injection only, γ-irradiation only and combination NP/γ treatment, with a post-randomization check to ensure that starting tumor size was similar across groups.

Control and treatment groups for the survival study are listed as follows: (i) untreated control group (n=17), (ii) NPCP-CTX control group (n=14; this treatment group received a total body iron oxide dose of 8.5 mg kg⁻¹, with an iron oxide concentration of injected solution of about 0.8 mg Fe/ml⁻¹ and a total injection volume of about 200 μL, and the final injection volume was adjusted based on exact animal weight), (iii) 10 Gy γ-irradiation control group (n=9) and (iv) combined NPCP-CTX+10 Gy treatment group (n=12). In group iv, radiation was delivered 1 hour after NPCP-CTX injection, with equivalent NP and radiation doses compared to control groups ii and iii, respectively. All injected solutions were prepared and conjugated to CTX on the day of injection. Injections were performed via tail vein puncture. Mice were monitored daily and euthanized when standard humane study endpoints were reached (e.g., 15% loss of body weight, development of severe neurological symptoms such as hunched posture, head tilt, and moribund appearance). Survival was monitored and analyzed using a Kaplan-Meier plot. Statistical analysis was performed using the log rank test.

### Magnetic Resonance Imaging

MRI was performed on a 14 Tesla (600 MHz) Bruker Avance III vertical-bore spectrometer. Isoflurane (Piramal Healthcare) in oxygen was supplied through a coil-integrated respiratory monitoring system (SA Instruments), with bite bar and ear bar restraints to maintain fixed head position. Respiratory rate was monitored to control depth of anesthesia, and a circulating water control bath maintained constant temperature.

Four hours of sequential T₂*-weighted imaging were performed to measure intracranial presence of NPCP and NPCP-CTX in healthy and tumor tissue. T₂*-weighted images were acquired every 10 min with a fast low angle shot (FLASH) pulse sequence in the coronal plane (TE 6.0 ms, TR 1000 ms, in-plane resolution 78×78 m², slice thickness 0.5 mm). Scan duration was approximately 4 min. Analysis of images was accomplished using the Paravision 5.1 analysis package (Bruker) and Osirix (Pixmeo) (A. Rosset, L. Spadola, O. Ratib, J. Digit. Imaging 17 (2004) 205-216). To account for inherent signal intensity changes due to inter-scan drift in gain, power, shim and tuning, all images were normalized based on the intensity of a water phantom located adjacent to the mouse head. Magnetic susceptibilities of NPCP and NPCP-CTX were tested in aqueous solution and were found to be identical.

Using groups of three mice from each experimental group (12 total), in vivo MRI of mice was performed once a week from the time of tumor implantation, to monitor the growth of the implanted GBM tumors (6 weeks total scanning). Whole-head coronal orientation T₂-weighted images were acquired using the rapid acquisition with refocused echoes (RARE) pulse sequence (TE 6.78 ms, TR 4000 ms, in-plane resolution 78×52 m², slice thickness 0.5 mm). Scan duration was approximately 4 min. 3D tumor volumes were calculated using Paravision 5.1 analysis software over the complete set of slices encompassing each tumor. Volumetric analysis was based on the T₂ signal intensity change between the tumor margin and normal brain. Volume was measured independently by three separate observers and averaged to account for human error in boundary estimation.

### Magnetic Resonance Spectroscopy

MRS was performed on mice from each of the treatment groups defined previously, 10 days after treatment. A ¹H point resolved spectroscopy (PRESS) scan sequence was used with water suppression (TR 2.5 s, TE 30 ms, SW 50 kHz, NA 480). For tumor MRS, the single 10 mm³ voxel was prescribed at the outer boundary of the tumor, maintaining the entirety of the voxel within tumor tissue, and avoiding the potentially necrotic central tumor region. For non-tumor MRS, the voxel was prescribed within contralateral cortical brain tissue, at a distance 2-3 mm from the primary tumor. Duration of each scan was approximately 20 min. Phase correction and apodization were carried out with the Paravision 5.1 analysis package. Spectral peak integrals were calculated using Gaussian/Lorentzian curve fitting software programs developed in MATLAB (The Mathworks, Inc).

### Iron Quantitation Assay

Quantification of cellular NPCP uptake in GBM6 was determined as described in O. Veiseh, J. W. Gunn, F. M. Kievit, C. Sun, C. Fang, J. S. Lee, M. Zhang, Small 5 (2009) 256-264. Briefly, 200,000 GBM6 cells were seeded per well in a 12-well plate and were grown for 24 hours at 37° C. in a humidified atmosphere with 5% CO₂. Cells were washed with PBS and incubated in cell culture medium containing NPCP at concentrations of 0, 25 and 100 μg ml⁻¹ for 2 hours at 37° C. After incubation, NPs not internalized were removed through three successive washes with PBS. Next, cells in the 12-well plates were lysed with 400 μl of 50 mM NaOH, vortexed for 30 s and neutralized with 300 μl 10 mM HCl. Iron content was to be normalized to cellular protein content. Protein quantification was determined through the Bradford assay by adding 6 μl of cell lysate solution to 300 μl of Bradford reagent in a 96-well plate and incubated for 10 min at room temperature. Absorbance was measured at 595 nm and protein amount was determined using a standard curve. To quantify the iron content of the lysate, 300 μl of iron-releasing reagent (0.7 M HCl, 2.25% KMnO₄ in deionized water) was added to 300 μl of cell lysate and incubated for 2 hours at 60° C. Samples were cooled to room temperature and 90 μl of ferrozine solution (1 M ascorbic acid, 2.5 μM ammonium acetate, 6.5 mM ferrozine, 0.135% neocuproine in deionized water) was added. Samples were incubated for 30 min at room temperature, and the absorbance at 562 nm was measured. The values were fit to a standard curve and normalized to protein content per cell.

### Nanoparticle Synthesis

NPs were produced by both thermal decomposition and co-precipitation approaches and were coated with various polymer formulations including crosslinked silane-poly(ethylene glycol) (PEG), amphiphilic phospholipid-grafted PEG (PLP), chitosan-grafted PEG (CP) and catechol modified CP (CCP). Thermal decomposition of Fe-oleate precursor produced hydrophobic oleic acid coated iron oxide NPs (IOOA). The oleic acid coating of IOOA was removed through a ligand exchange process using succinic anhydride-functional silane followed by grafting of PEG to the silanized iron oxide surface resulting in water soluble iron oxide NPs coated with a silanized PEG monolayer (IOSPM). Alternatively, IOOA was mixed with PLP in non-polar organic solvent, followed by a slow increase in polarity of the solution to drive self-assembly through hydrophobic interactions to create iron oxide NPs coated with PLP (IOPLP). IOSPM and IOPLP maintained the same iron oxide core size and structure, however, IOPLP contained a hydrophobic region between the core and the outer PEG layer, spatially separating water from the iron oxide core. Iron oxide NPs coated with CP (NPCP) and CCP (IOCCP) as well as maghemite core NPs coated with CP were synthesized by co-precipitation of iron chlorides in aqueous solution. IOCCP and NPCP were synthesized with 2:1 ratio of Fe³⁺ to Fe²⁺, producing magnetite cores. However, IOCCP was produced utilizing CCP, where the catechol serves as the capping agent. Maghemite NPs were produced identically to NPCP (magnetite) but using only Fe³⁺ in the co-precipitation synthesis.

### Transmission Electron Microscopy (TEM) and Selected Area Electron Diffraction (SAED) Analysis of Nanoparticles

Samples were prepared by depositing dilute nanoparticle solutions onto carbon film-coated copper grids. TEM and SAED images were acquired with an FEI Tecnai F20 TEM (Hillsboro, OR) operating at 200 kV. Rings in the reciprocal space resulting from the electron beam diffraction were analyzed using ImageJ software.

### Fourier Transform Infrared (FTIR) Spectroscopy Analysis of Nanoparticles

Nanoparticles were synthesized, then lyophilized to obtain dry samples. The samples were mixed into a KBr pellet at 0.2 wt %. The FTIR spectra of the various nanoparticle samples were obtained using Nicolet 6700 FTIR Spectrometer (ThermoFisher, Waltham, MA).

### Assessment of Stability of NPCP and NPCP-CTX in PBS

NPCP and NPCP-CTX were prepared and dispersed in PBS (pH 7.4). Hydrodynamic sizes of the nanoparticles were measured using a DTS Zetasizer Nano (Malvern Instruments, Worcestershire, UK). The samples were maintained in the same measurement cuvette and measurements were made once a day for 7 days.

### Cellular Uptake Assay for NPCP and NPCP-CTX

Fluorescence microscopy was used to assess cellular uptake of NPCP and NPCP-CTX. NPCP was dispersed in HEPES buffer (pH 7.4). Cyanine5-N-hydroxysuccinimide (Cy5-NHS) was added to the NPCP solution at a mass ratio of 1:100 Cy5:NPCP to obtain Cy5-labelled NPCP (NPCP-Cy5). CTX was then conjugated onto NPCP-Cy5 using the method provided in the main text to obtain Cy5-labelled NPCP-CTX (NPCP-Cy5-CTX). SF763 cells were seeded and incubated for 12 h with cell culture media containing 10, 25, or 50 μg mL⁻¹ of NPCP-Cy5 or NPCP-Cy5-CTX, after which the cells were imaged using a Nikon TE300 inverted fluorescence microscope (Nikon, Tokyo, Japan).

### Pathologic Tissue Analysis

Whole brains of athymic nude mice inoculated with GBM6 cells were removed through necropsy 10 days after treatment administration (9 weeks after tumor inoculation) and preserved in 10% formalin for 48 h, followed by embedding in paraffin wax. The formalin-fixed paraffin-embedded tumor tissues were cut into 4 μm thick sections and dried at 70° C. for 30 min on standard glass slides. For H&E staining, the sections were stained with hematoxylin (Polyscience, Inc., Warrington, PA) for 40 s and with eosin (Sigma-Aldrich, 5 St Louis, MO) for 30 s. Immunohistochemical staining for Ki67 (Abcam, ab15580) and phospho-histone H2A.X (Ser-139) (Cell Signaling, #9718) antibodies (Ab) was performed at Children's Hospital Los Angeles using the Leica Bond RXm™ automated staining processor (Leica Biosystems, Buffalo Grove, IL). 4 μm thick sections were dewaxed and antigen retrieval was performed in Bond Rx system with Epitope Retrieval 2 (pH 9) for 20 min. Subsequent 30 min incubation was performed with rabbit polyclonal anti-Ki-67 Ab and rabbit monoclonal pHistone H2A.X (Ser-139) Ab at concentrations of 1:800 and 1:1500, respectively. The Leica DAB detection kit was used following manufacturer's protocol. All slides were subsequently scanned with the Aperio ScanScope XT system (Leica Biosystems, Buffalo Grove, IL) at 20× magnification. The automated digital images were analyzed and scored using Qupath 0.2.3 software. Ki-67 and γH2AX were analyzed over 10 random fields of view. Violin plots were generated for each condition, using both standard labeling index, as well as the histopathology (H)-score method.

TABLE 1
Physiochemical properties of NPs.

| | | | | Iron Oxide | |
|---|---|---|---|---|---|
| | Core Size | Core | Core | Surface | |
| NP | (nm) | Morphology | composition | Ligand | Polymer Coating |
| IOSPM | 12.6 ± 0.9 | Spherical | Fe₃O₄ | Ester | Crosslinked |
| | | | | | Silane-PEG |
| IOCCP | 4.9 ± 0.8 | Rough | Fe₃O₄ | Catechol | Chitosan-PEG |
| | | Spherical | | | |
| NPCP | 4.4 ± 1.3 | Rough | Fe₃O₄ | Chitosan | Chitosan-PEG |
| | | Spherical | | | |
| Maghemite NP | 3.8 ± 0.7 | Rough | Fe₂O₃ | Chitosan | Chitosan-PEG |
| | | Spherical | | | |
| IOPLP | 12.8 ± 0.8 | Spherical | Fe₃O₄ | Carboxylic | Oleic |
| | | | | acid | acid/Phospholipid- |
| | | | | | PEG |

TABLE 2
Diffraction data d-spacings in Å, based on the
rings and standard atomic spacing for Fe₃O₄ and Fe₂O₃.

| | 1 | 2 | 3 | 4 | 5 | 6 |
|---|---|---|---|---|---|---|
| NPCP | 3.050 | 2.564 | 2.121 | 1.717 | 1.641 | 1.495 |
| NPCP- | 3.002 | 2.547 | 2.115 | 1.721 | 1.628 | 1.489 |
| CTX | | | | | | |
| IOCC P | 2.981 | 2.548 | 2.112 | —* | 1.627 | 1.486 |
| IOSPM | 2.985 | 2.528 | 2.120 | 1.719 | —* | 1.487 |
| IOPLP | 2.994 | 2.539 | 2.119 | 1.722 | —* | 1.491 |
| Fe₃O₄** | 2.967 | 2.532 | 2.100 | 1.715 | 1.616 | 1.485 |
| MCP | 2.964 | 2.525 | 2.101 | —* | —* | 1.478 |
| Fe₂O₃** | 2.953 | 2.518 | 2.087 | 1.705 | 1.607 | 1.476 |

*Intensity of the diffracted beam was too low to identify the d-spacing.
**Reference iron oxide powder diffraction data (S. Asuha, S. Zhao, H. Y. Wu, L. Song, O. Tegus, Journal of Alloys and Compounds 472 (2009) L23-L25).

While illustrative embodiments have been illustrated and described, it will be appreciated that various changes can be made therein without departing from the spirit and scope of the invention.

1. A method for targeted radiation therapy in a subject, comprising:
(a) administering to a subject an iron oxide nanoparticle having an iron oxide core, a biocompatible coating surrounding the core, and a targeting agent associated with the coating, to provide a site in the subject having accumulated iron oxide nanoparticles; and
(b) applying γ- and/or x-ray irradiation to the subject at the site having accumulated iron oxide nanoparticles to produce photoelectrons at the site.

2. A method for producing photoelectrons at a select site in a subject, comprising:
(a) administering to a subject an iron oxide nanoparticle having an iron oxide core, a biocompatible coating surrounding the core, and a targeting agent associated with the coating, to provide a site in the subject having accumulated iron oxide nanoparticles; and
(b) applying γ- and/or x-ray irradiation to the subject at the site having accumulated iron oxide nanoparticles to produce photoelectrons at the site.

3. A method for treating a cancer in a subject, comprising:
(a) administering to a subject an iron oxide nanoparticle having an iron oxide core, a biocompatible coating surrounding the core, and a targeting agent associated with the coating, to provide a site in the subject having accumulated iron oxide nanoparticles; and
(b) applying γ- and/or x-ray irradiation to the subject at the site having accumulated iron oxide nanoparticles to produce photoelectrons at the site, wherein the site is a cancerous tumor.

4. The method of claim 3, wherein the photoelectrons are Auger photoelectrons.

5. The method of claim 3, wherein the targeting agent selectively delivers the iron oxide nanoparticle to the site.

6. The method of claim 3, wherein the targeting agent is chlorotoxin.

7. The method of claim 3, wherein the targeting agent selectively delivers the iron oxide nanoparticle to the cancerous tumor.

8. The method of claim 3, wherein the cancerous tumor is a solid tumor.

9. The method of claim 3, wherein the cancerous tumor is a brain tumor of any pathology.

10. The method of claim 3, wherein the cancerous tumor is a primary brain tumor.

11. The method of claim 3, wherein the cancerous tumor is a neuroectodermal tumors.

12. The method of claim 3, wherein the cancerous tumor is a tumor of the breast, kidney, liver, lung, lymphoma, ovarian, pancreas, prostate, bone, cervix, colon, or throat.

13. The method of n claim 3, wherein the iron oxide nanoparticle is administered to the subject intravenously.

14. The method of claim 3, wherein the iron oxide core comprises magnetite.

15. The method of claim 3, wherein the coating is effective to disperse the iron oxide nanoparticles and have thicknesses in the range from about 1 to about 100 nm.

16. The method of claim 3, wherein the coating comprises a silanized poly(ethylene glycol) (PEG) monolayer (IOSPM) or a chitosan-PEG (CP) copolymer (NPCP) layer.

17. The method of claim 3, wherein the nanoparticle has a diameter from about 5 to about 200 nm.

18-19. (canceled)
